(19) United States
(12) Patent Application Publication
(10) Pub. No.: US 2017/0319359 A1
(43) Pub. Date: Nov. 9, 2017

(54) ENDOVASCULAR DOCKING APPARATUS AND METHOD

(71) Applicant: Manish MEHTA, Mechanocville, NY (US)

(72) Inventor: Manish Mehta, Mechanicville, NY (US)

(21) Appl. No.: 14/784,426

(22) PCT Filed: Apr. 16, 2014
(86) PCT No.: PCT/US14/34429

§ 371 Date: Oct. 14, 2015

Related U.S. Application Data
(60) Provisional application No. 61/812,523, filed on Apr. 16, 2013.

(51) Int. Cl. A61F 2/82; A61F 2/07; A61F 2/954; A61F 2/06

(52) U.S. Cl. CPC A61F 2/82; A61F 2/954; A61F 2/07; A61F 2220/0075; A61F 2002/067

(57) ABSTRACT

Exemplary embodiments of apparatuses and methods of providing an endovascular′dock within a blood vessel are provided. An apparatus for vascular surgery can be provided, having an external tubular graft capable of expansion and configured to be placed within a sheath in an unexpended state, a first tubular structure provided internally within the external tubular graft and configured for placement of a graft therein, and a second tubular structure provided internally within the external tubular graft and configured for placement of a graft therein. Stent grafts can be provided along each tubular structure to a corresponding blood vessel such that blood flow is provided to the blood vessel from the apparatus within the stent grafts to each blood vessel, blocking the blood flow directly from the aneurysm.

### CROSS-REFERENCE TO RELATED APPLICATION(S)

This application relates to and claims priority from U.S. Patent Application Ser. No. 61/812,523 filed Apr. 16, 2013, the entire disclosure of which is hereby incorporated herein by reference.

### FIELD OF THE DISCLOSURE

The present disclosure relates to exemplary embodiments of endovascular docking apparatuses and methods, and more particularly, to exemplary embodiments of endovascular docking apparatuses and methods for providing blood flow between blood vessels.

### BACKGROUND INFORMATION

The aorta is the main blood vessel that carries blood from the heart to the rest of the body and can be approximately similar in size to a large garden hose. The aorta wraps around the heart and travels through the chest (where it is known as the thoracic aorta) into the lower abdomen (where it becomes the abdominal aorta). Along the way, the aorta gives rise to blood vessels that supply circulation to all parts of the body. An aneurysm is a progressive weakening and ballooning of the blood vessel wall, a condition that commonly affects the thoracic and abdominal aorta, and the iliac arteries. If undiagnosed and untreated, an aneurysm can rupture, which can result in internal bleeding and in some instances, death.

Conventional vascular grafts are commonly used for treating aneurysms, and can be composed of flexible tubes of woven or knitted polyethylene terephthalate (e.g., Dacron®) or polytetrafluoroethylene (“PTFE”). These vascular grafts require surgical approach, and exposure of the aneurysms as well as the normal healthy aorta at proximal and distal ends of the aneurysm. The grafts are sewn into the healthy aorta above and below the aneurysm to divert blood flow. These procedures can require surgery, and expose the patients to a significant risk for a higher morbidity and mortality, increased length of convalescence and lengthy recovery periods. This can be a problem to patients that are older, sicker, and/or have more risk factors. Furthermore, when aortic and aorto-iliac aneurysms involve the thoracic arch great vessels, the abdominal visceral vessels or the pelvic internal iliac arteries, adjunctive hybrid surgical procedures can often be required to achieve aneurysm exclusion. Currently, stent grafts designed to address these issues are not available.

Vascular stent grafts composed of polyethylene terephthalate or PTFE are devices that are supported with stents and packaged into delivery sheaths, and can also be used for treating aortic aneurysms. These delivery sheaths are inserted into the aorta from remote access sites such as the femoral or iliac arteries, and advanced from within the aneurysms and deployed to anchor the healthy aorta proximal and distal to the aortic aneurysm. As a result, the aneurysm can be excluded from the circulation and depressurized.

Although stent grafts can offer a minimally invasive solution to treating aortic aneurysms and limit the morbidity and mortality, currently available devices have many limitations and can only be used to treat approximately half of all aortic aneurysms. Furthermore, the aorta starts form the aortic valve in the heart and ends at the iliac arteries in the mid-abdomen, and the iliac arteries extend down to the level of the groin and transition into the femoral arteries. Along the path, the thoracic aorta gives rise to all great vessels that supply the upper extremities, head and neck, and the abdominal aorta gives rise to all visceral vessels that supply all vital organs in the abdomen. The iliac arteries give rise to vessels that supply the pelvic organs. Although the aorta is a single organ and all aortoiliac segments are affected by aneurysmal disease, currently available stent grafts are fundamentally designed to target only independently treated aneurysms that involve the thoracic aorta, or the abdominal aorta and the iliac arteries.

Currently available stent grafts are single system tubular systems, modular bifurcated systems, fenestrated, or branched stent graft systems. All the currently available stent grafts have limitations because of, e.g., their inability to treat thoracic and abdominal aortic aneurysms as a whole, rather only having the ability to treat select sections of the thoracic and abdominal aortic aneurysms. This can often result in repeat and multiple procedures to adequately exclude the entire extent of the aortic aneurysms. Currently there is no single device that can treat all thoracic aortic, abdominal aortic and iliac aneurysms, while preserving blood flow to all the vital arch and visceral side-branches. With extensive aneurysms, a significant challenge has been to exclude the thoracic aortic aneurysm while providing flow to the great vessels, as well as to exclude the abdominal aorta while providing perfusion to the visceral vessels.

Furthermore, current fenestrate and branched stent grafts have many limitations, including but not limited to: 1) procedure complexity that prohibits routine alignment to stent graft fenestrations and branches to the thoracic arch and abdominal visceral and pelvic internal iliac arteries, resulting in excessive device manipulation that can lead to embolization, resulting in stroke, paraplegia, renal failure, bowel ischemia, lower extremity ischemia and various other organ malperfusion; 2) inadequate construct to accommodate most proximal aortic neck landing zones, particularly when treating aortic aneurysms involving the thoracic aortic arch, or the abdominal visceral vessels; and 3) inadequate aortic neck seal resulting in increased incidence of endoleaks, risks of end organ malperfusion with fenestration and branch stent graft thrombosis.

At least one of the objects of the exemplary embodiments of the present disclosure is to reduce or address the deficiencies and/or limitations of the prior art procedures and systems described herein above, by providing an endovascular docking method and system configured to treat aneurysms that does not suffer from the inabilities of current stent grafts.

### SUMMARY OF EXEMPLARY EMBODIMENTS OF THE PRESENT DISCLOSURE

At least some of the above described problems can be addressed by exemplary embodiments of the system, method and computer accessible medium according to the present disclosure. For example, using such exemplary embodiments, it is possible to provide an apparatus for vascular surgery, comprising an external tubular graft capable of expansion and configured to be placed within a sheath in an unexpanded state, a first tubular structure provided internally within the external tubular graft and configured for placement of a graft therein, and a second tubular structure provided internally within the external tubular graft and configured for placement of a graft therein. The external tubular graft can comprise a fabric made of polytetrafluoroethylene or polyethylene terephthalate.

The apparatus can further comprise one or more stents provide along a tubular wall of the external tubular graft. The one or more stents can comprise steel, nickel, titanium or nitinol. The one or more stents can be provided in one of a spiral, straight, circular or zigzag configuration.

The first tubular structure can have a larger diameter than the second tubular structure. The apparatus can further comprise one or more stents provided along a tubular wall of the first tubular structure, and one or more stents provided along a tubular wall of the second tubular structure. The tubular wall of the first tubular structure and the tubular wall of the second tubular structure can be attached to an inner portion of the tubular wall of the external tubular graft. The first and second tubular structures can have approximately a same height as the external tubular graft.

The apparatus can further comprise a third tubular structure provided internally within the external tubular graft and configured for placement of a graft therein, and a fourth tubular structure provided internally within the external tubular graft and configured for placement of a graft therein, wherein the first tubular structure has a larger diameter than the second, third and fourth tubular structures, and the second, third and fourth tubular structures have approximately a same diameter.

Using such exemplary embodiments, it is also possible to provide a method of providing an apparatus for vascular surgery, comprising providing an external tubular stent graft having a tubular wall and configured to be placed within a sheath in an unexpanded state, providing a first tubular structure within the external tubular stent graft and having a tubular wall attached to the tubular wall of the external tubular stent graft, and configured for placement of a graft therein, and providing a second tubular structure within the external tubular stent graft and having a tubular wall attached to the tubular wall of the external tubular stent graft, and configured for placement of a graft therein.

The method can further comprise providing stents on the tubular walls of the first and second tubular structures. The method can further comprise providing a third tubular structure within the external tubular stent graft and having a tubular wall attached to the tubular wall of the external tubular stent graft, and configured for placement of a graft therein, and providing a fourth tubular structure within the external tubular stent graft and having a tubular wall attached to the tubular wall of the external tubular stent graft, and configured for placement of a graft therein. The first tubular structure can have a larger diameter than the second, third and fourth tubular structures, and the second, third and fourth tubular structures can have approximately a same diameter.

Using such exemplary embodiments, it is also possible to provide a method of performing vascular surgery, comprising providing an endovascular dock within a sheath, the endovascular dock comprising an external tubular stent graft having a tubular wall, a first tubular structure provided within the tubular wall of the external tubular stent graft, and a second tubular structure provided within the tubular wall of the external tubular stent graft, retracting the sheath to dock the endovascular dock within a wall of a first blood vessel,

providing a first stent graft having a first end within the first tubular structure and a second end within a wall of a second blood vessel such that blood flow is substantially restricted to within the first stent graft between the first stent graft and the second blood vessel, and providing a second stent graft having a first end within the second tubular structure and a second end within a wall of a third blood vessel to provide blood flow between the second stent graft and the third blood vessel such that blood flow is substantially restricted to within the second stent graft between the second stent graft and the third blood vessel. The method can further comprise providing a polymer to fill a void between the external walls of the first and second tubular structures and the internal wall of the external tubular stent graft of the endovascular dock.

The first end of the first stent graft can expand to conform to the shape of the first tubular structure and the second end of the first stent graft expands to conform to the shape of the wall of the second blood vessel, and the first end of the second stent graft expands to conform to the shape of the second tubular structure and the second end of the second stent graft expands to conform to the shape of the wall of the third blood vessel. The first stent graft can be provided by obtaining access to the first tubular structure through the wall of the second blood vessel. The second stent graft can be provided by obtaining access to the second tubular structure through the wall of the third blood vessel.

The method can, further comprise providing a third stent graft having a first end within the second end of the first stent graft, and a second end having a first and second tubular wall, a first tubular wall being provided within a wall of a fourth blood vessel such that blood flow is substantially restricted to between the first stent graft and the fourth blood vessel, and a second tubular wall being provided within a wall of a fifth blood vessel such that blood flow is substantially restricted to between the first stent graft and the fifth blood vessel.

Using such exemplary embodiments, it is also possible to provide a system for providing an endovascular dock within a blood vessel, comprising an endovascular dock having an external tubular stent graft, a first tubular structure provided internally within the external tubular stent graft and configured for placement of a first graft therein, a second tubular structure provided internally within the external tubular stent graft and configured for placement of a second graft therein, a sheath for housing the endovascular dock within the sheath, and a top portion connected to a distal end of the sheath, wherein the endovascular dock is configured to be placed within a distal end of the sheath in a non-expanded state and is configured to expand when the sheath is retracted from the top portion. The top portion can comprise a nose cone having a hole at a top portion for insertion of a wire.

The system can further comprise a first catheter having one end within the sheath and extending through the first tubular structure into the top portion, a second catheter having one end within the sheath and extending through the second tubular structure into the top portion, a first guide wire provided within the first catheter, a second guide wire provided within the second catheter, and a center shaft provided having one end within the sheath and extending within the external tubular stent graft and attached to the top portion.

The system can further comprise a third tubular structure provided internally within the external tubular stent graft and configured for placement of a third graft therein, wherein the center shaft is provided through the third tubular structure within the external tubular stent graft. The system can further comprise one or more radio opaque markings along the external tubular stent graft in a location corresponding to the first and second tubular structures.

These and other objects, features and advantages of the present disclosure will become apparent upon reading the following detailed description of embodiments of the present disclosure, when taken in conjunction with the appended claims.

### BRIEF DESCRIPTION OF THE DRAWINGS

The foregoing and other exemplary objects of the present disclosure will be apparent upon consideration of the following detailed description, taken in conjunction with the accompanying exemplary drawings and claims, in which like reference characters refer to like parts throughout, and in which:

FIG. 1 illustrates a perspective view of an endovascular dock according to an exemplary embodiment of the present disclosure;

FIGS. 2(a)-2(b) illustrate perspective views of an endovascular dock according to exemplary embodiments of the present disclosure;

FIG. 3 illustrates a cross-sectional view of an endovascular dock according to an exemplary embodiment of the present disclosure;

FIGS. 4(a)-4(i) illustrate a method of providing an endovascular dock to treat a thoracic aortic aneurysm and a thoracic aortic arch aneurysm according to an exemplary embodiment of the present disclosure;

FIGS. 5(a)-5(h) illustrate a method of providing an endovascular dock to treat an abdominal aortic aneurysm/juxtarenal abdominal aortic aneurysm/thoracoabdominal aortic aneurysm according to an exemplary embodiment of the present disclosure;

FIGS. 6(a)-6(c) illustrate a method of providing an endovascular dock to treat an abdominal aortic aneurysm/juxtarenal abdominal aortic aneurysm/thoracoabdominal aortic aneurysm according to an exemplary embodiment of the present disclosure;

FIGS. 7(a)-7(e) illustrate a method of providing an endovascular dock to treat a thoracic aortic aneurysm/abdominal aortic aneurysm/thoracoabdominal aortic aneurysm according to an exemplary embodiment of the present disclosure;

FIGS. 8(a)-8(c) illustrate a method of providing an endovascular dock to treat an abdominal aortic aneurysm and an iliac aortic aneurysm according to an exemplary embodiment of the present disclosure;

FIGS. 9(a)-9(d) illustrate a delivery system for delivering an endovascular dock according to an exemplary embodiment of the present disclosure;

FIGS. 9(e)-9(g) illustrate another embodiment of a delivery system for delivering an endovascular dock according to an exemplary embodiment of the present disclosure; and

FIG. 9(h) illustrates endovascular dock having radio opaque markings according to an exemplary embodiment of the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the subject disclosure.

### DETAILED DESCRIPTION OF EXEMPLARY EMBODIMENTS OF DISCLOSURE

Exemplary embodiments of the methods and systems of the present disclosure will now be described with reference to the figures.

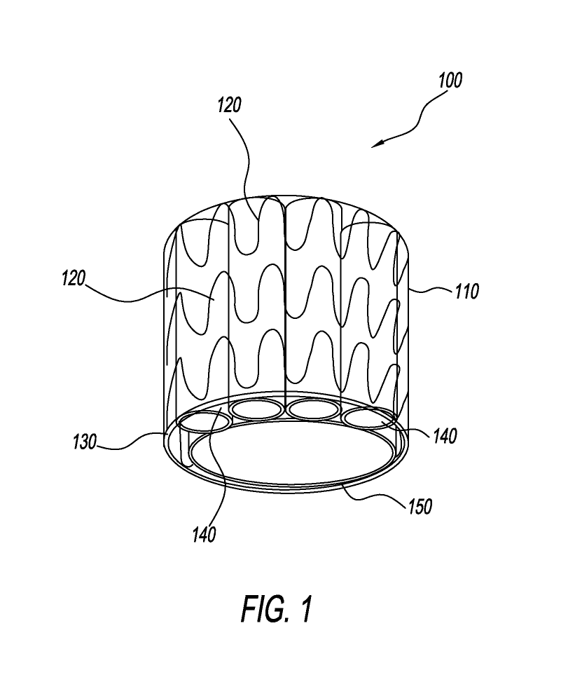

FIG. 1 illustrates a perspective view of an endovascular dock 100 according to an exemplary embodiment of the present disclosure. An endovascular dock 100 can be provided that can create a branch point within the lumen of any blood vessel (e.g., artery, vein) where it is placed. The endovascular dock 100 can have an external tubular graft 110, which can be a flexible self-expanding stent supporting prosthetic graft. The tubular graft 110 can comprise one or more stents 120, which can be made of steel, nickel, titanium, Nitinol, or other similar material, and a fabric 130, which can be PTFE, an expanded PTFE (“e-PTFE”), polyethylene terephthalate or other similar fabric. The stents 120 can be provided along a circumference of the fabric 130, and can be placed along an inner wall of the external tubular graft 110 in order to expand the fabric 130, or the fabric 130 can be imbedded inside the stents 120. Other embodiments are also possible, where the stents 120 can be placed in the middle of two layers of fabric 130 that make up the tubular graft 110, and the present disclosure is not limited to any particular embodiment.

The stents 120 can have a spiral configuration, but can also have different shapes/configurations, such as straight, zigzag or circular configurations. The stents 120 can be sutured to the fabric 130. As shown in FIG. 1, three stents 120 are provided along the length of the endovascular dock 100. However, more or less stents 120 can be used, which can depend on, e.g., the length of the endovascular dock 100. The proximal most portion of the stents 120 can have outward projected barbs for active fixation along its circumference for attachment to the fabric 130.

One or more smaller caliber tubular structures 140 can be provided within the tubular graft 110. These tubular structures 140 can comprise a fabric which can provide an outer layer along a circumference of the tubular structures 140. As shown in FIG. 1, four tubular structures 140 can be provided within the tubular graft 110, but more or less may be used and the present disclosure is not limited to any particular number. The tubular structures 140 can be adjacent to one another or separated from one another within the tubular graft 110. A larger caliber tubular structure 150 can also be provided within the tubular graft 110. The tubular structures 140 and/or the larger tubular structure 150 can optionally have one or more stents similar to the tubular graft 110 along their circumference (not shown) to provide for expansion of the tubular structures, but may not be required.

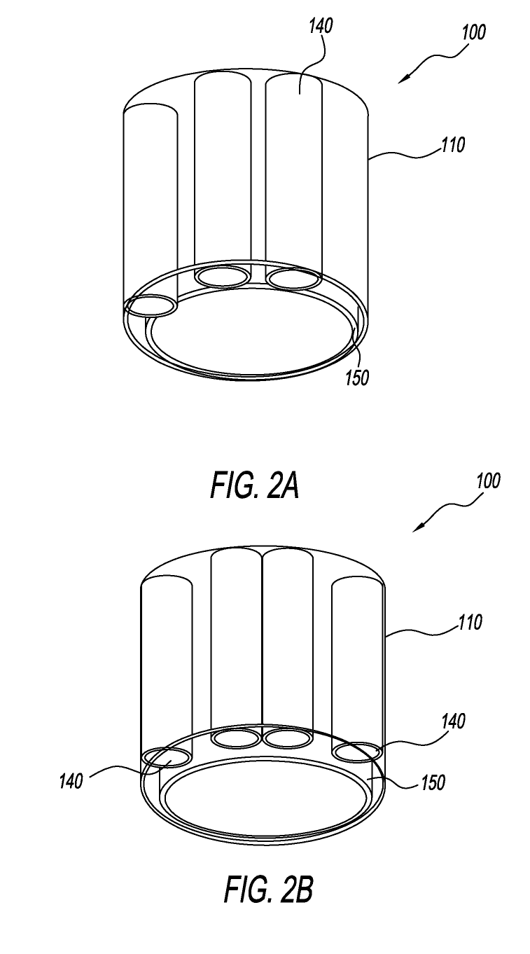

FIGS. 2(a) and 2(b) illustrate perspective views of an endovascular dock 100 according to exemplary embodiments of the present disclosure. The tubular graft 110 can have within it, along a luminal surface, one or more smaller caliber tubular structures 140 which are aligned along the tubular graft 110. In some exemplary embodiments, two to five smaller caliber tubular structures 140 can be provided. As seen in FIG. 2(a), a tubular graft 110 is provided having three smaller caliber tubular structures 140 separated from each other, and a larger tubular structure 150. As seen in FIG. 2(b), a tubular graft 110 is provided having four smaller caliber tubular structures 140, where two center tubular structures are proximate to each other and the two outside tubular structures are separated from the rest. A larger caliber tubular structure 150 can also be provided within the tubular graft 110. The smaller caliber tubular structures 140 and the larger caliber tubular structure 150 can have an outer wall that is attached to an inner wall of the tubular graft 110 to keep it in place. The tubular structures can be sewn to or attached via any method to the tubular graft 110. Stents 120 (not shown) can also be provided on the tubular graft 110, and can also be provided on the smaller tubular structures 140 and the larger tubular structure 150. The present disclosure is not limited to any particular number of smaller or larger caliber tubular structures or position within the tubular graft 110.

The length of the tubular graft 110 can be anywhere between 1 centimeter to 20 centimeters, and the diameter can be between 1 to 10 cm. The present disclosure is not limited to any particular length or diameter. In some exemplary embodiments, the length of the tubular graft 110 can be approximately three to approximately seven centimeters, and the diameter can be between approximately two to approximately five centimeters. The length of the smaller caliber tubular structures 140 and the larger caliber tubular structure 150 can be the same length as the tubular graft 110, as shown in FIG. 1, or can be shorter than the tubular graft 110. The diameter of the smaller caliber tubular structures and the larger caliber tubular structures can be less than the diameter of the tubular graft 110, and can range in size depending on the arteries being treated. The diameter of the larger tubular structure 150 can range from approximately 1 cm to approximately 5 cm, and the diameter of the smaller tubular structures 140 can range from approximately 0.5 cm to approximately 2 cm. The diameters and lengths are exemplary embodiments, and the present disclosure is not limited to any particular length or diameters of these elements.

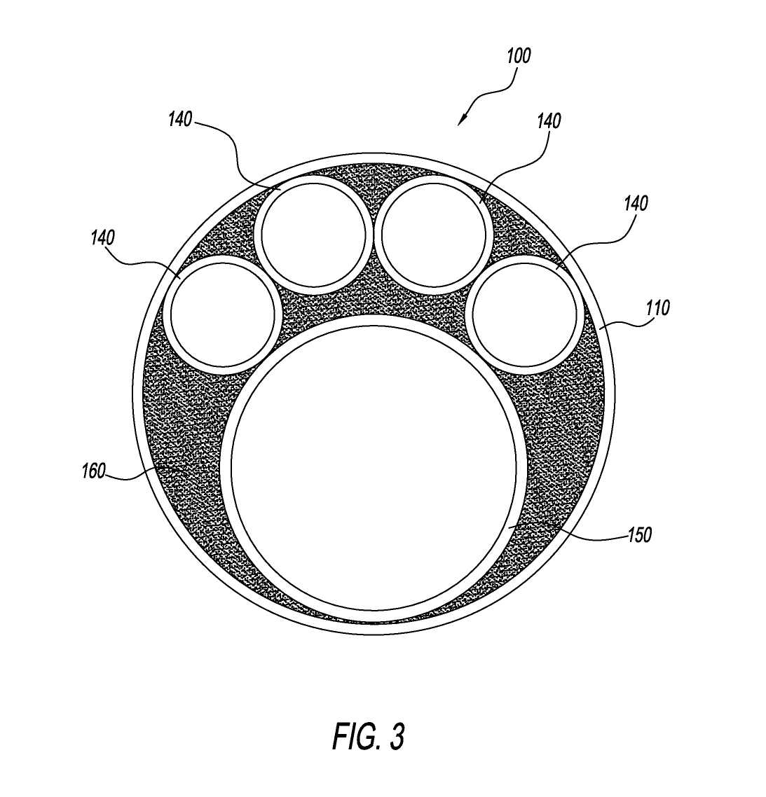

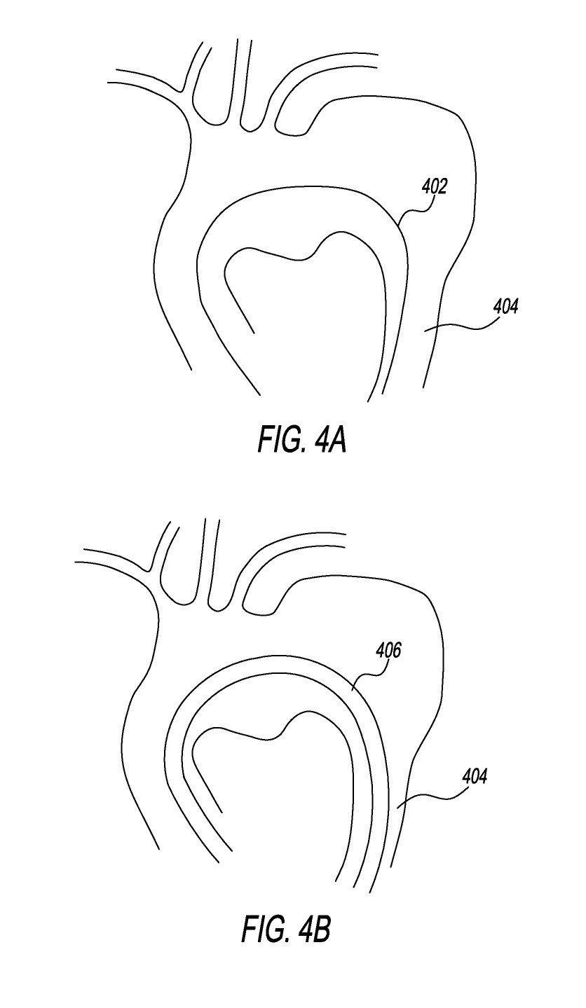

FIG. 3 illustrates a cross-sectional view of an endovascular dock 100 according to an exemplary embodiment of the present disclosure. Smaller tubular structures 140 and a larger tubular structure 150 can be provided within the tubular graft 110 of the endovascular dock 100. A polymer 160 can be provided within the space between the smaller tubular structures 140 and larger tubular structure 150. The polymer 160 can be added prior to deployment, or can be added once docked within the body, as will be explained below. Other materials besides polymers can also be used, such as but not limited to a polymer fill, EPTFE, polyethylene terephthalate or other similar material.

As shown in FIG. 3, the tubular graft 110 can have one larger tubular structure 150, and smaller tubular structures 140. The smaller tubular structures 140 and larger tubular structure can be tubular stent grafts. The tubular graft 110 can act as a dock for tubular stent grafts in the thoracic aorta or bifurcated stent grafts in the abdominal aorta, and the smaller caliber tubular structures 140 and larger caliber tubular structure 150 can be a dock for single piece tubular stent grafts, as will be explained below.

The number of smaller tubular structures 140 and/or larger tubular structures 150 can depend on the location being treated. For example, if the endovascular dock 100 is deployed in the ascending thoracic aorta, and further stent grafts are needed to treat thoracic aortic aneurysms that involve the thoracic arch, then all vital thoracic arch vessels supplying the upper extremities and the brain can be preserved. The stent grafts used to preserve these blood vessels can be docked within the endovascular dock 100 proximally and within the healthy blood vessel supplying the brain and the upper extremities distally. In this example, only a short proximal landing zone may be needed, which can be approximately two centimeters, and the endovascular dock length can be anywhere from approximately four centimeters to approximately 6 centimeters, although the endovascular dock of the present disclosure is not limited to any particular length or diameter.

The endovascular dock 100 can be packaged into a sheath that would be used to deliver it to various locations within the lumen of a blood vessel, such as the aorta or iliac arteries. The constrained endovascular dock 100 can travel within a packaged sheath delivery system that would travel over a wire to various locations intraluminally. To deploy the endovascular dock 100, the sheath delivery system within which the device is housed can be retracted back to deliver the device which can fully open to oppose the luminal side of the blood vessel. Some components of small barbs (approximately 2-3 mm) can be provided along the outer portion of the fabric 130, that penetrate and anchor within the blood vessel wall to keep the endovascular dock in place and can prevent any upward or downward displacement. Once deployed, the endovascular dock 100 can provide continuous uninterrupted flow through all tubular structures within the device 100, and prevent blood flow (by the, e.g., polymer 160) in any other portions of the endovascular dock 100. The space between the tubular structures 140/150 and the tubular stent graft 110 can be filled by a polymer 160, or a polymer 160 can be infused after. A polymer fill, EPTFE, polyethylene terephthalate or other similar material could also be used. In some embodiments, the endovascular dock 100 can be provided such that the tubular structures 140/150 are provided such that there are substantially no openings between the tubular structures 140/150 in the tubular stent graft 110.

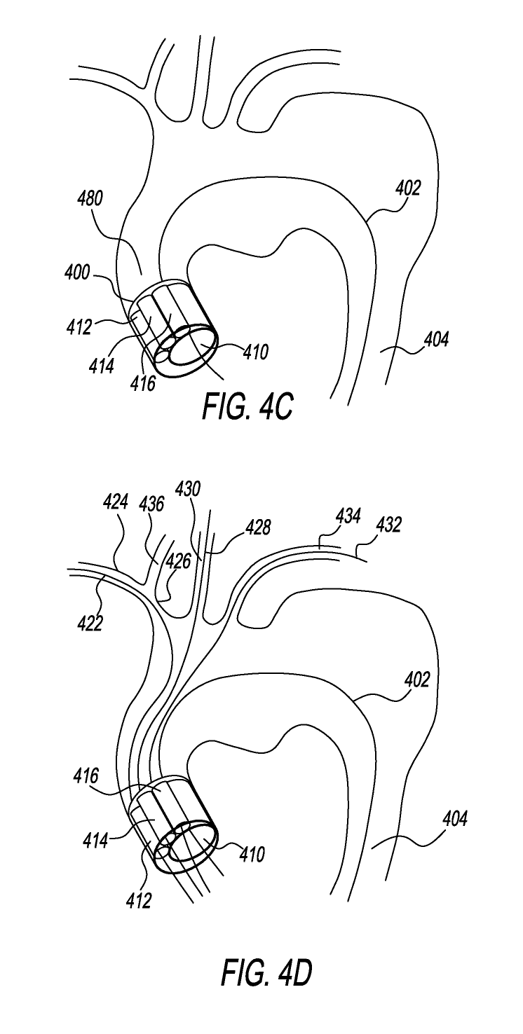

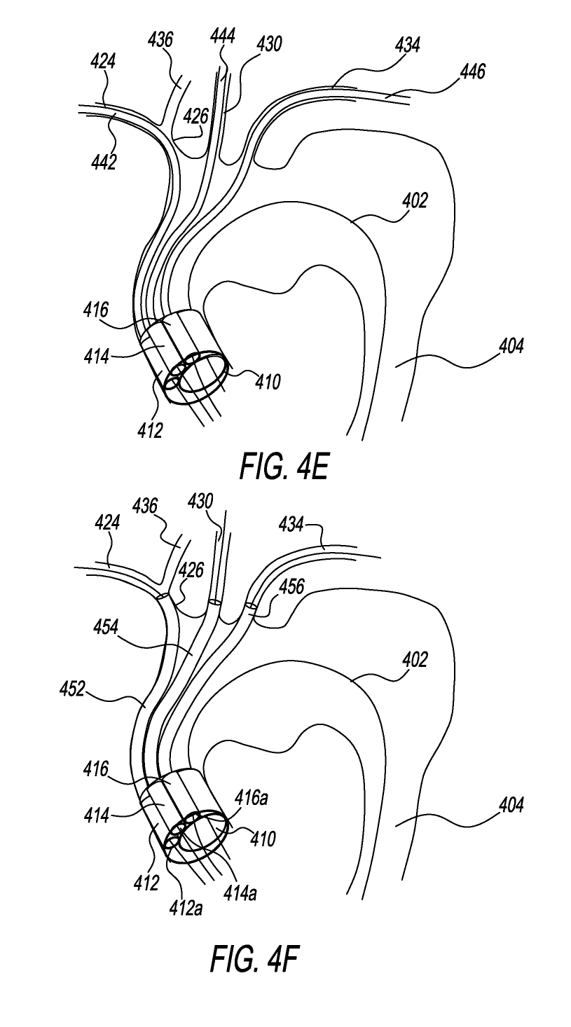

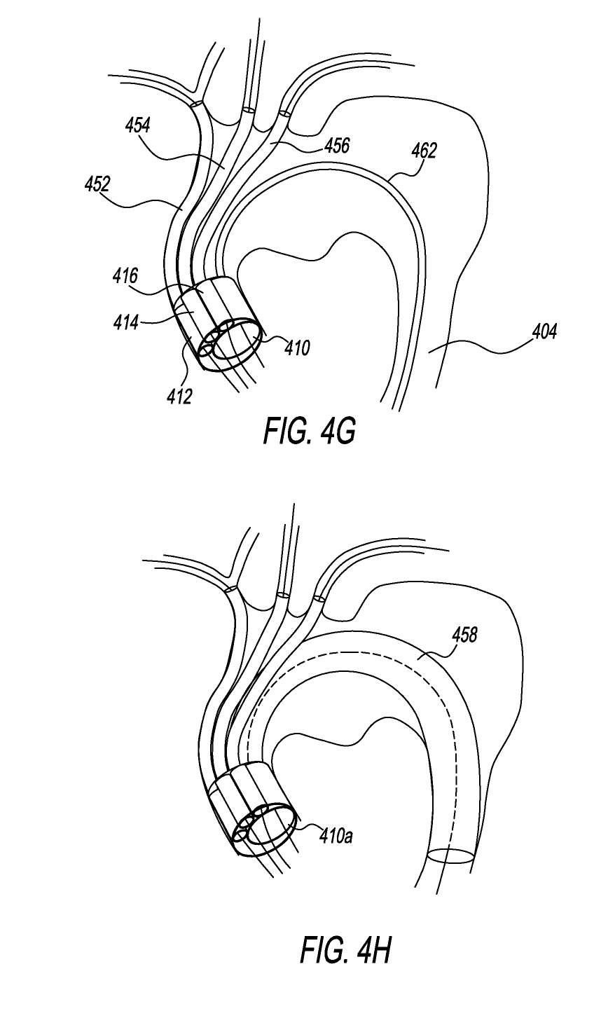

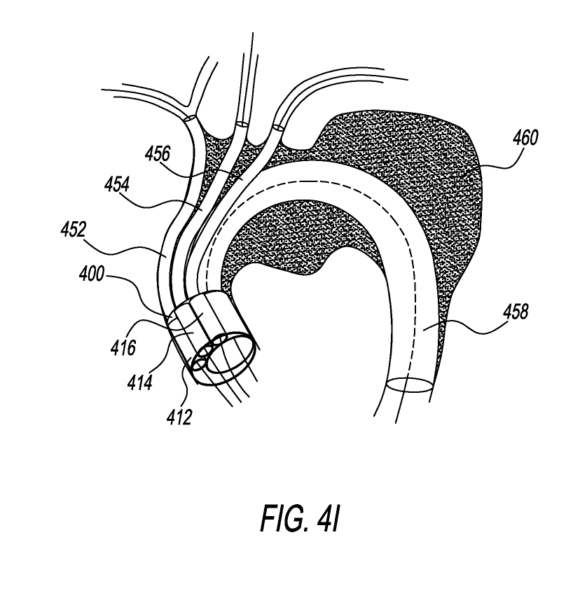

FIGS. 4(a)-4(i) illustrate a method of providing an endovascular dock to treat a thoracic aortic aneurysm and a thoracic aortic arch aneurysm according to an exemplary embodiment of the present disclosure. In this exemplary embodiment, an endovascular dock is provided to treat a thoracic aortic aneurysm that includes the thoracic arch, which is the part of the aorta that has the great arch vessels (i.e., the subclavian, carotid and innominate arteries).

As shown in FIG. 4(a), a wire 402 can be inserted through an entrance 404 via access from the femoral arteries in the groin. Then, as shown in FIG. 4(b), a sheath 406 can be brought over the wire 402 containing an endovascular dock within the sheath 404. The endovascular dock 400 is placed within the sheath 406 such that the wire 402 is placed within a larger tubular structure 410 in the endovascular dock 400. The size of the endovascular dock 400 used can vary depending on the size of the aorta being treated. As shown in FIG. 4(c), the sheath 406 is pulled back deploying the endovascular dock 400 at the location desired, in this embodiment, the ascending thoracic aorta 480. Barbs can be provided along the outer circumference of the fabric of the endovascular dock 400 to hold the endovascular dock 400 in place at the ascending thoracic aorta 480 such that there is no displacement of the endovascular dock 400 once deployed. The endovascular dock 400 can have stents (as described in FIG. 1) such that the endovascular dock 400 can be self-expanding once deployed, to block the entire wall of the thoracic aorta 480 as shown in FIG. 4c.

As shown in FIG. 4(d), once the endovascular dock 400 is deployed, a wire 422 can be brought in through the right subclavian artery 424 through the innominate artery 426 into the tubular structure 412 within the endovascular dock 400. The wire 422 could also be brought down the right carotid artery 436 through the innominate artery 426 into the tubular structure 412 within the endovascular dock 400. A wire 428 can be brought in through the left cartotid artery 430 into the tubular structure 414 within the endovascular dock 400. A wire 432 can be brought in through the left subclavian artery 434 into the tubular structure 416 within the endovascular dock 400.

As shown in FIG. 4(e), a sheath 442 can be brought over wire 422 through the right subclavian artery 424 through the innominate artery 426 into the tubular structure 412. A sheath 444 can be brought over wire 428 through the left cartotid artery 430 into the tubular structure 414. A sheath 446 can be brought over wire 432 in through the left subclavian artery 434 into the tubular structure 416.

As shown in FIG. 4(f), the sheath 442 can then be retracted deploying a stent graft 452 within the tubular structure 412. The stent graft 452 can be deployed such that a first end of the stent graft 452 is placed within the tubular structure 412. The first end of the stent graft 452 can be placed anywhere within the tubular structure 412, and can preferably be placed at or proximate to a distal end 412a, to ensure a proper and secure fit. The length of the stent graft 452 can be selected such that a second end of the stent graft 452 is placed within the wall of the innominate artery 426. The stent graft 452 can be self-expanding and can adhere to the walls of the innominate artery 426 such that any blood flow outside the tubular structure 412 is prevented. Placing the second end of the stent graft 452 at the walls of the innominate artery 426 can allow blood flow to be provided to both the right subclavian artery 424 and the right carotid artery 436, preventing the need for two different stent grafts for both these arteries.

Similarly, the sheath 444 is retracted deploying a stent graft 454 within the tubular structure 414. The stent graft 454 can be deployed such that a first end of the stent graft 454 is placed at or proximate to a distal end 414a of the tubular structure 414. The length of the stent graft 454 can be selected such that a second end of the stent graft 454 is placed within the wall of the left carotid artery 430. The stent graft 454 can be self-expanding such that it adheres to the wall of the left carotid artery 430. The sheath 446 is retracted deploying a stent graft 456 within the tubular structure 416. The stent graft 456 can be deployed such that a first end of the stent graft 456 is placed at or proximate to a distal end 416a of the tubular structure 416. The length of the stent graft 456 can be selected such that a second end of the stent graft 456 is placed within the wall of the left subclavian artery 434. The stent graft 456 can be self-expanding such that it adheres to the wall of the left subclavian artery 434.

As shown in FIG. 4(g), a sheath 462 is brought in over the wire 402 from the entrance 404 via access from the femoral arteries in the groin, through the larger tubular structure 410. As shown in FIG. 4h, the sheath 462 is retracted deploying a stent graft 458 within the tubular structure 410. The stent graft 458 can be deployed such that a first end of the stent graft 458 is placed at or proximate to a distal end 410a of the tubular structure 410. The length of the stent graft 458 can be selected such that a second end of the stent graft 458 is placed within the wall of the entrance 404 of the thoracic aorta. The stent graft 458 can be self-expanding such that it adheres and blocks the entrance 404 of the thoracic aorta. A polymer (not shown) can be provided within the spaces between the tubular structures 410, 412, 414 and 416. The polymer can be provided prior to deployment or after docking. As shown in FIG. 4i, the endovascular dock 400 and stent grafts 452, 454, 456 and 458 can provide for blood flow within the arteries and restrict any blood flow to the aneurysm 460.

The wires and sheaths may be provided in different manners and configurations, and there is no particular order that may be necessary as to which arteries to block first. The present disclosure contemplates multiple variations of the docking devices and methods of using the docking devices.

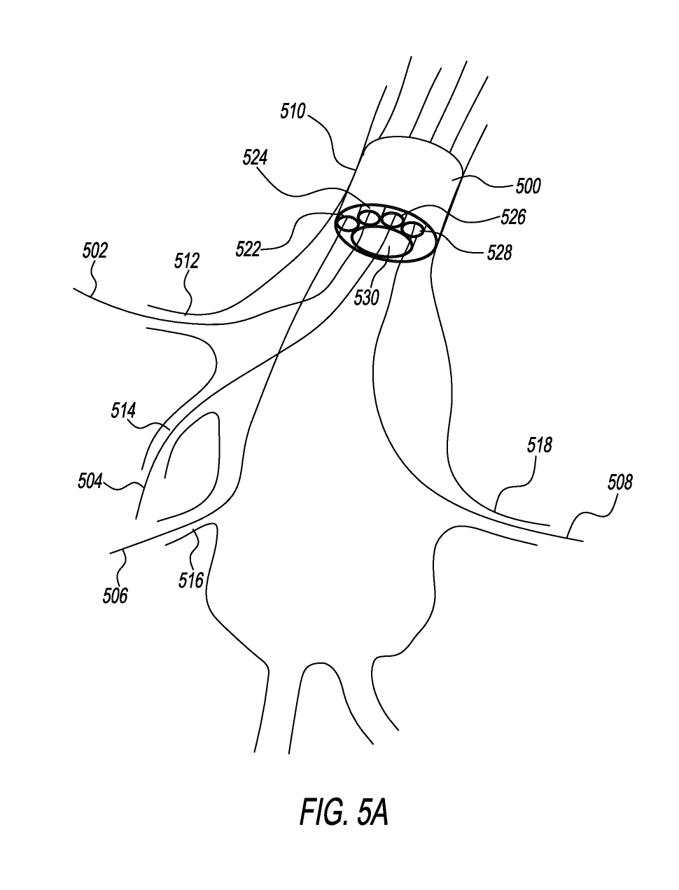

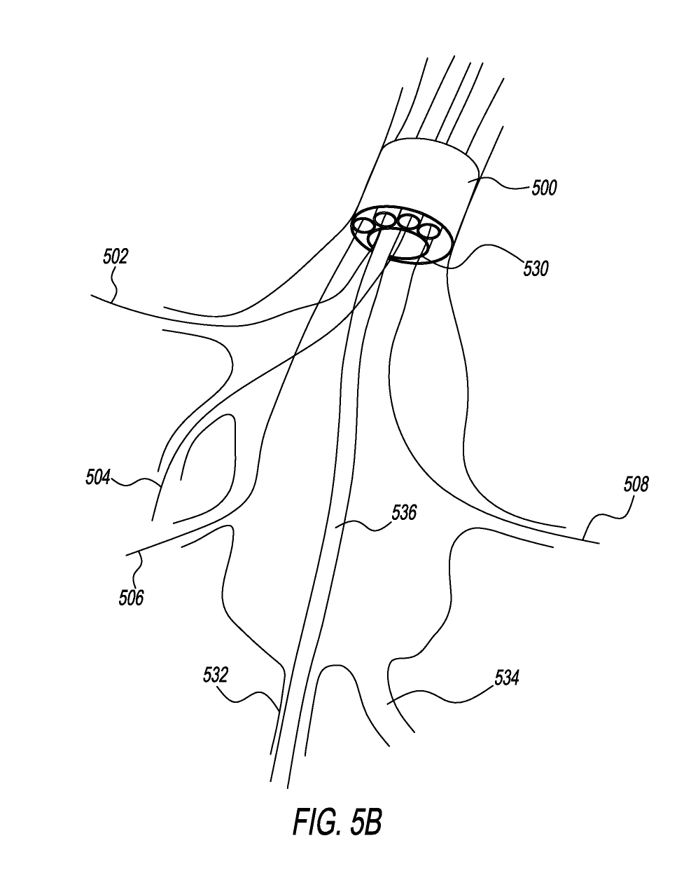

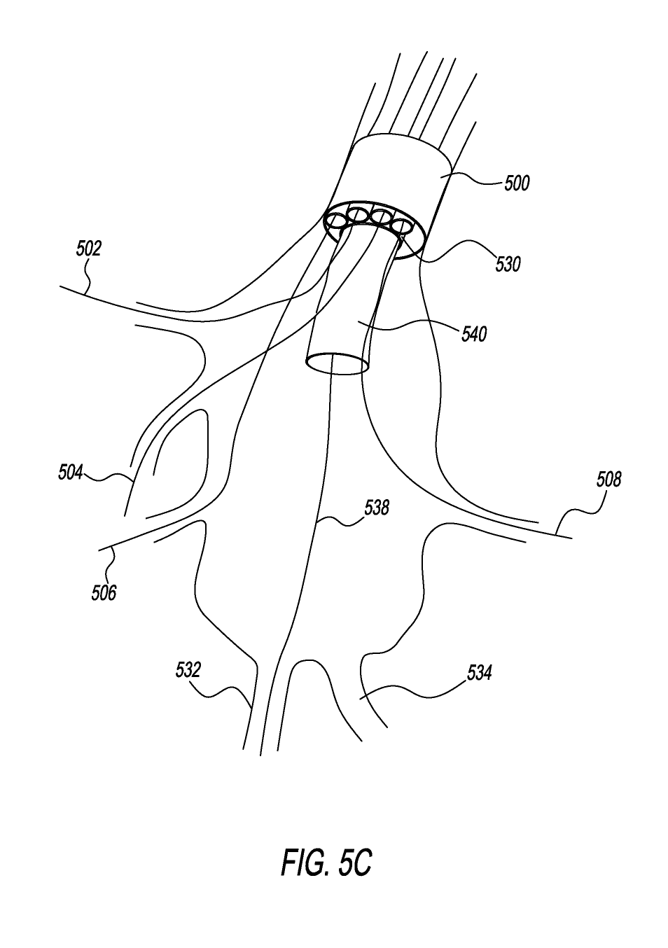

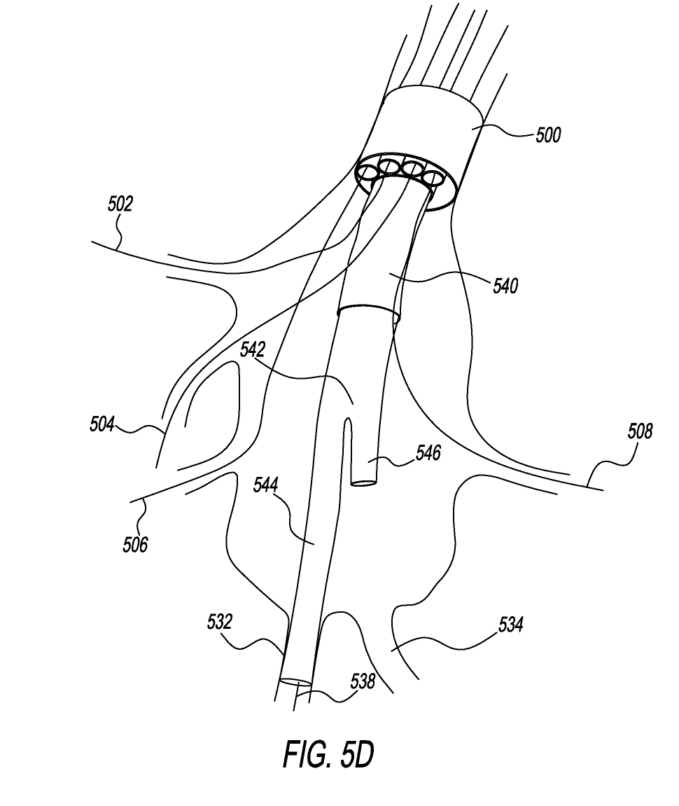

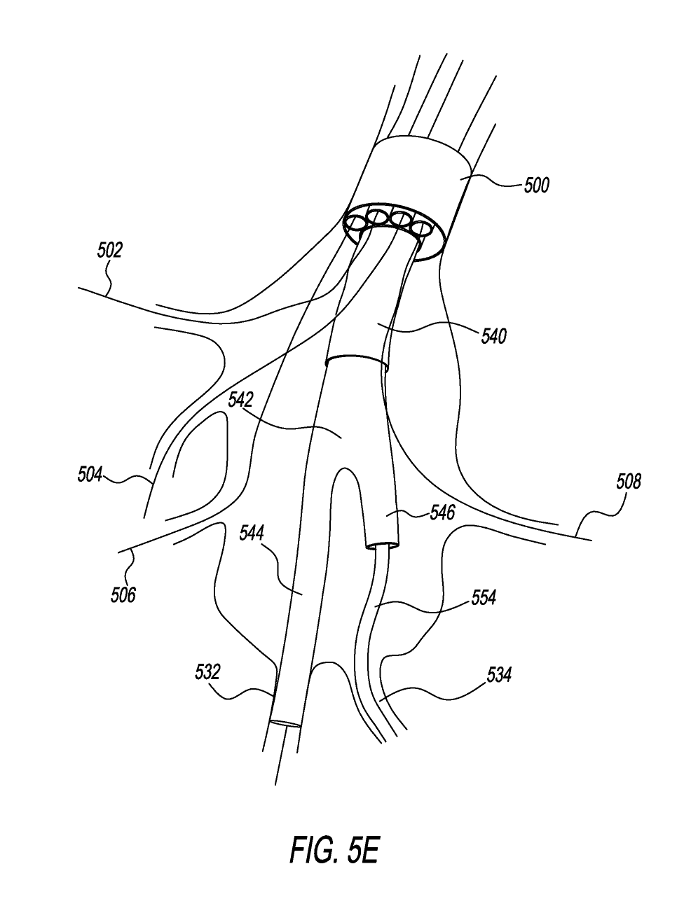

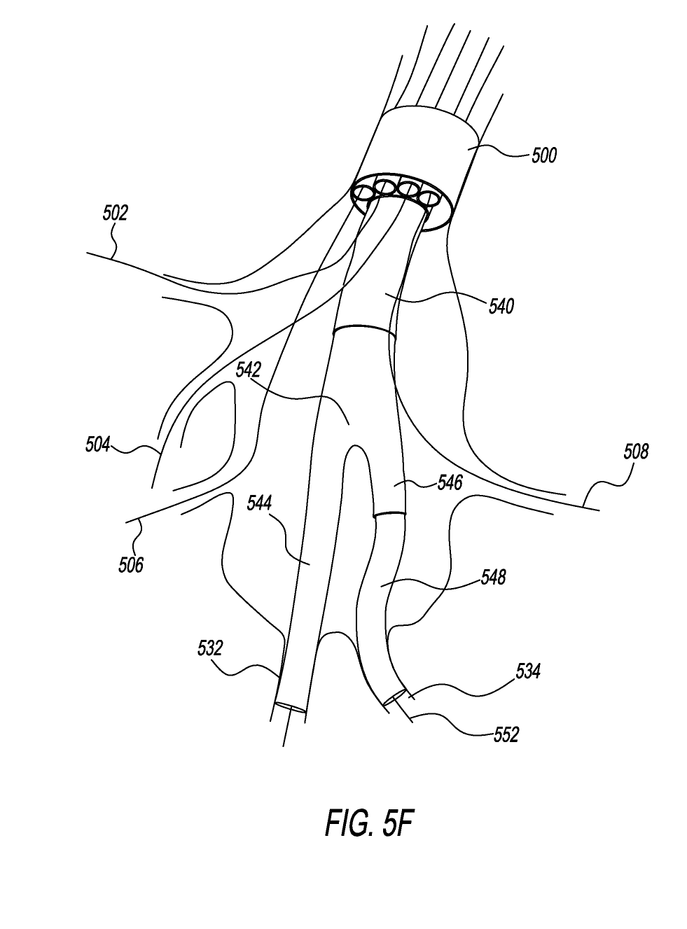

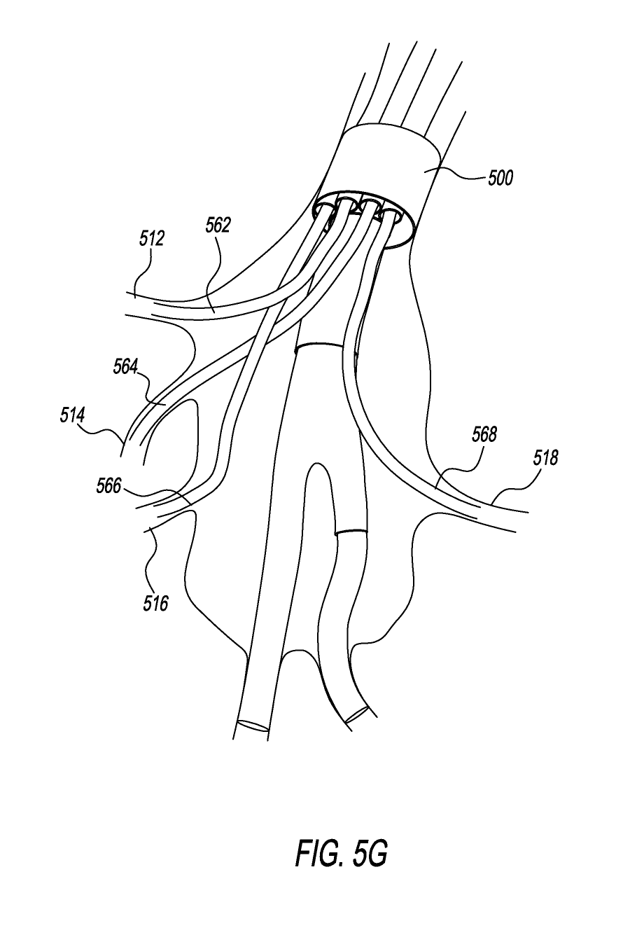

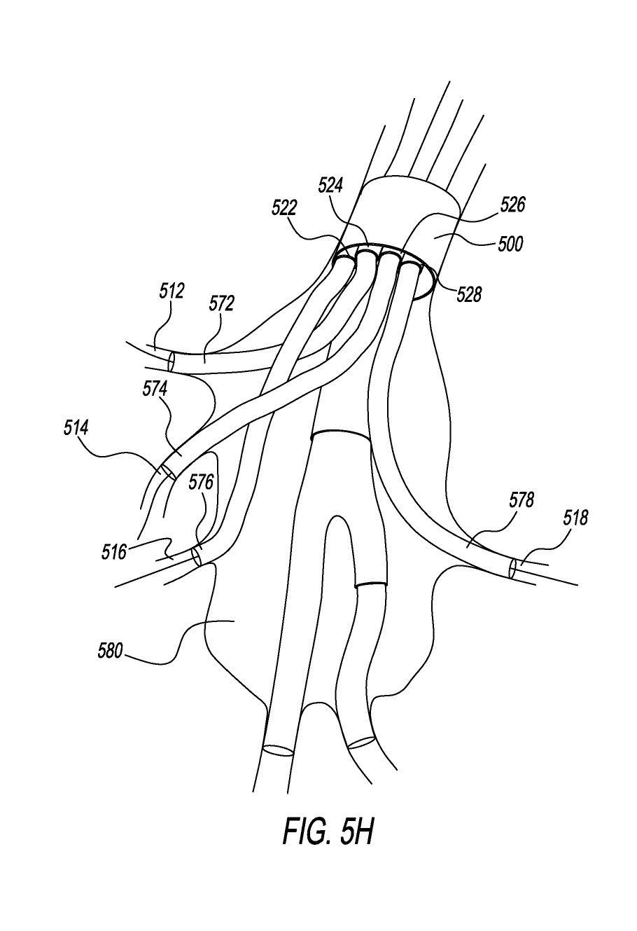

FIGS. 5(a)-5(h) illustrate a method of providing an endovascular dock to treat an abdominal aortic aneurysm/juxtarenal abdominal aortic aneurysm/thoracoabdominal aortic aneurysm according to an exemplary embodiment of the present disclosure. Initially, as shown in FIG. 5(a), a dock 500 can be placed within the thoracic aorta 510. Wires 502, 504, 506 and 508 can be brought in from visceral arteries 512, 514, 516 and 518, respectively. These wires can be brought in, e.g., from access via the arm or the top of the stomach. The wire 502 can be brought through tubular structure 524, wire 504 can be brought through tubular structure 526, wire 506 can be brought through tubular structure 522 and wire 508 can be brought through tubular structure 528. There is no particular order required as to which wire is brought in first or through which tubular structure to go through.

As shown in FIG. 5(b), a wire 538 can be brought in from iliac artery 532 through a larger tubular structure 530 in the tubular docking device 500. A sheath 536 can then be brought in over the wire 538 through the iliac artery 532 and through the larger tubular structure 530. When the sheath 538 is pulled back, a stent graft 540 can be deployed from the sheath 538 and installed within the larger tubular structure 530, as shown in FIG. 5(c). The stent graft 540 can be self-expanding such that it completely seals the inner circumference of the larger tubular structure 530. A sheath can then be deployed over wire 538 through the stent graft 540, and a stent graft 542 can then be deployed within the stent graft 540, as shown in FIG. 5(d). The stent graft 542 can be self-expanding such that it completely seals the inner circumference of the stent graft 540. The stent graft 542 can have a first portion 544 that includes a bottom portion that seals the wall of the iliac artery 532, preventing blood flow from outside the circumference of the first portion 544 of the stent graft 542. The first portion 544 of the stent graft 542 can be self-expanding to completely seal the wall of the iliac artery 532 to prevent such blood flow.

As shown in FIG. 5(e), a wire 552 can be brought through iliac artery 534 through a second portion 546 of the stent graft 542, and then a corresponding sheath 554 is brought overt the wire 552. As shown in FIG. 5(f), when the sheath 554 is pulled back, a stent graft 548 can be deployed within the second portion 546 of the stent graft 542, such that a top portion of the stent graft 548 self-expands to seal the inner circumference of the second portion 546 of the stent graft 542, and a bottom portion of the stent graft self-expands to seal an inner circumference of the walls of the iliac artery 534.

As shown in FIG. 5(g), sheaths 562, 564, 566 and 568 are brought in over the wires 502, 504, 506 and 508, respectively, and through the corresponding smaller tubular structures in the docking device 500. The sheaths 562, 564, 566 and 568 are then withdrawn to deploy stent grafts 572, 574, 576 and 578, respectively, which can be self-expanding such that a top portion of the stent grafts blocks the inner circumferences of the corresponding tubular structures 522, 524, 526 and 528. The bottom portions of the stent grafts 572, 574, 576 and 578 can block the walls of the visceral arteries 512, 514, 516 and 518, respectively. The lengths of the stent grafts 572, 574, 576 and 578 can be selected such that they provide the required length between the wall of the visceral artery and the tubular structures in the docking device 500. As shown in FIG. 5(h), the docking device 500 and stent grafts provided within the docking device can provide blood flow from the visceral and iliac arteries to the thoracic aorta 510, and block any blood flow to the aneurysm 580.

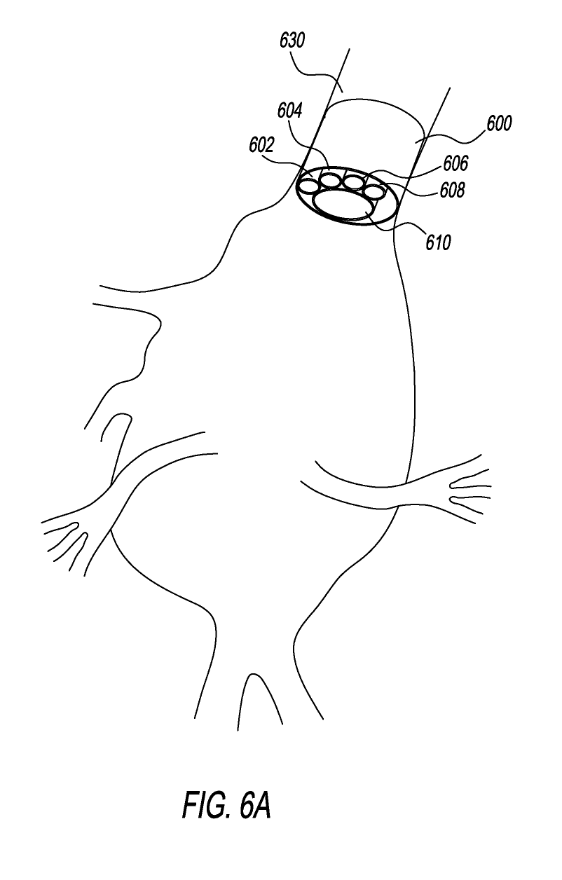

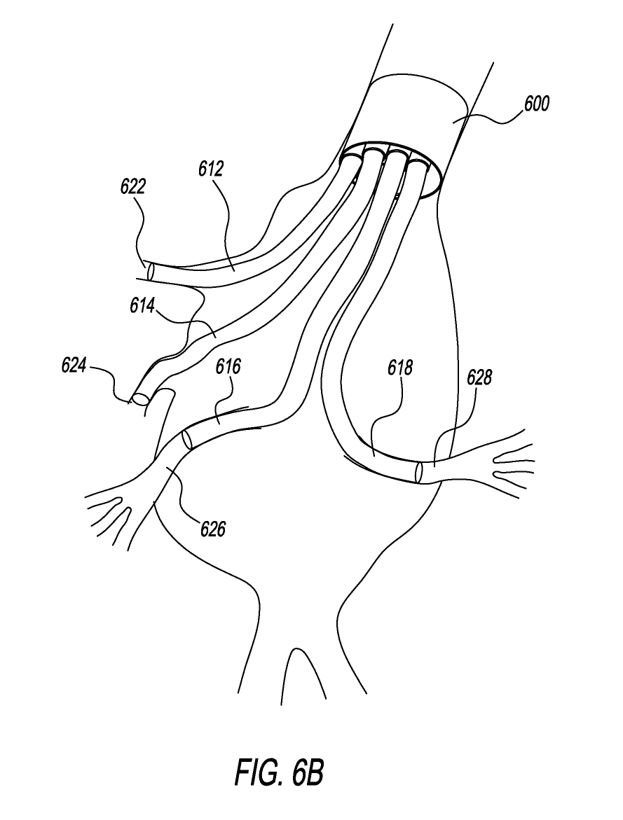

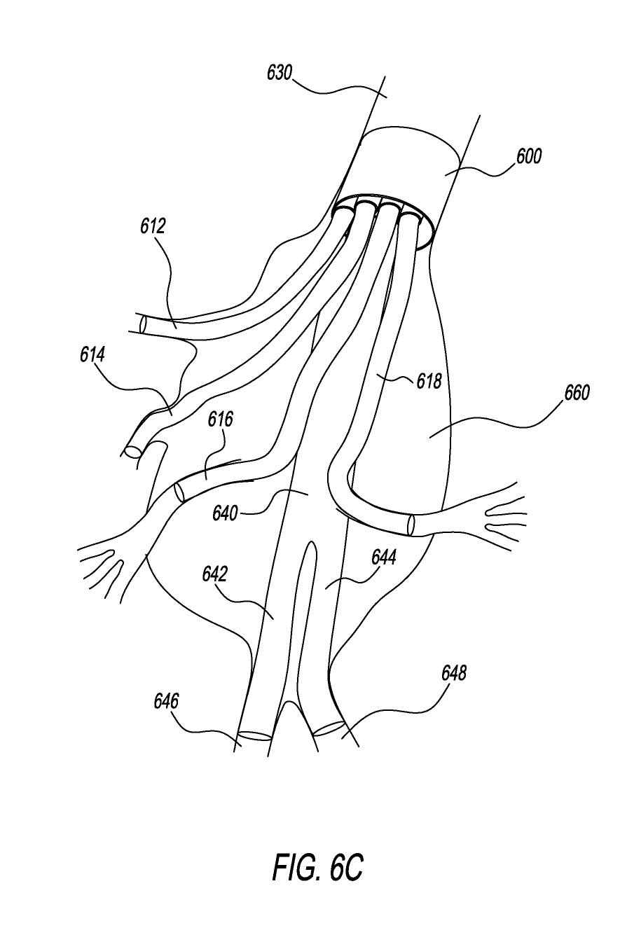

FIGS. 6(a)-6(c) illustrate a method of providing an endovascular dock to treat an abdominal aortic aneurysm/juxtarenal abdominal aortic aneurysm/thoracoabdominal aortic aneurysm according to an exemplary embodiment of the present disclosure. Initially, as shown in FIG. 6(a), a docking device 600 can be deployed within a sheath and installed as shown. The docking device 600 can be self-expanding to block the wall of the thoracic aorta 630. The docking device 600 can have four smaller caliber tubular structures 602, 604, 606 and 608, and a larger caliber tubular structure 610. As shown in FIG. 6(b), stent grafts 612, 614, 616 and 618 can be installed with one end in the smaller caliber tubular structures 602, 604, 606 and 608, and the other end blocking the walls of the visceral arteries 622, 624, 626 and 628 (using wires and sheaths as described with respect to FIGS. 5(a)-5(h)). Then, using either the entrance at iliac artery 646 or iliac artery 648, a wire and sheath (not shown) can be used to install a stent graft 640 within the larger caliber tubular structure 610. The stent graft 640 can be self-expanding such that a top portion of the of the stent graft 640 fit the inner circumference of the larger caliber tubular structure 610. The stent graft 640 can have a first portion 642 and a second portion 644. The first portion 642 of the stent graft 640 can be self-expanding to expand into the wall of the iliac artery 646, and the second portion 644 of the stent graft 640 can be self-expanding to expand into the wall of the iliac artery 648. The stent grafts and the docking device 600 can provide for blood flow from the thoracic aorta 630 to the visceral arteries 622, 624, 626 and 628, and the iliac arteries 646 and 648, while preventing blood flow to the aneurysm 660.

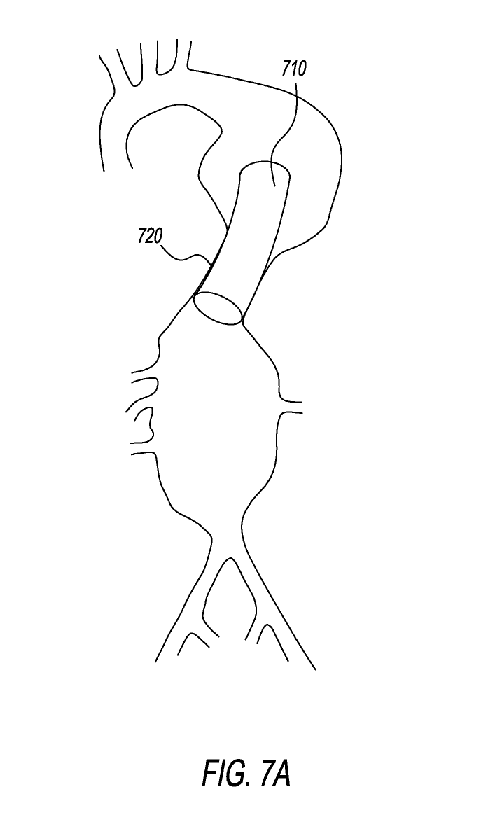

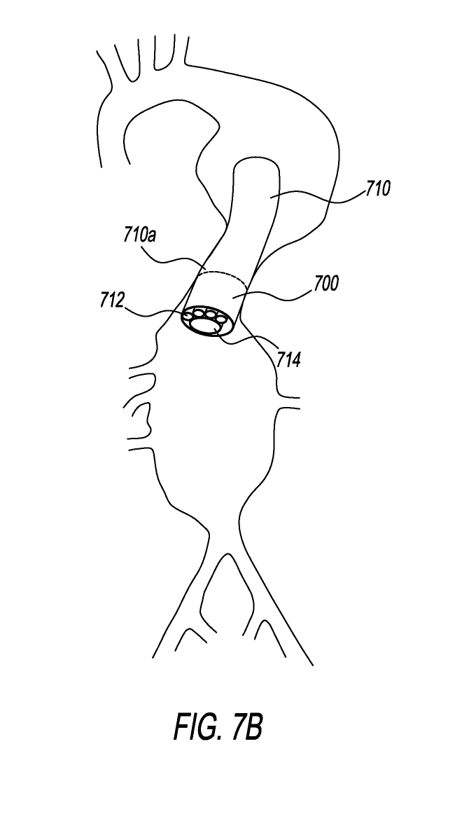

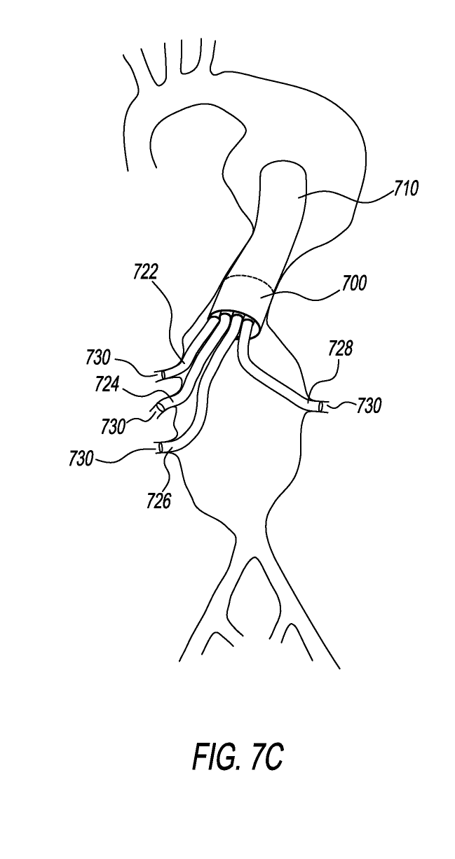

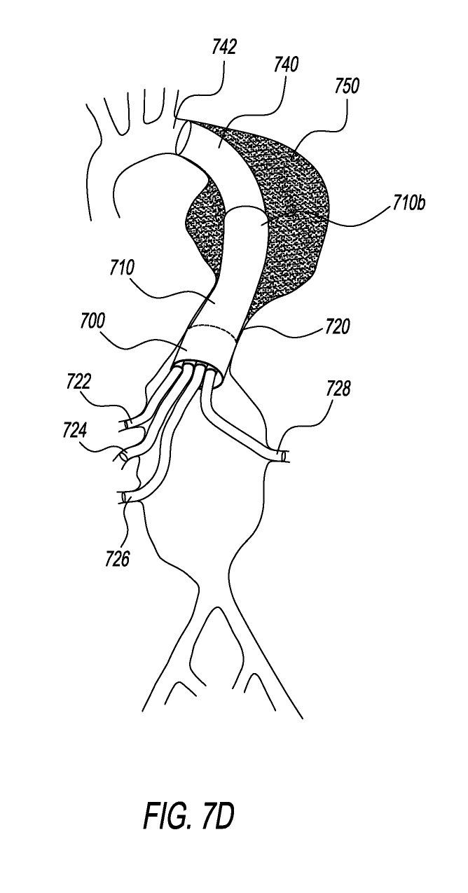

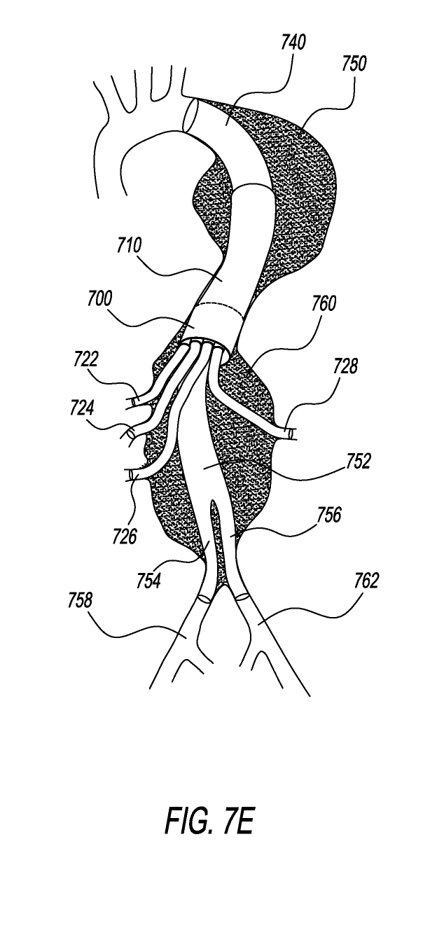

FIGS. 7(a)-7(e) illustrate a method of providing an endovascular dock to treat a thoracic aortic aneurysm/abdominal aortic aneurysm/thoracoabdominal aortic aneurysm according to an exemplary embodiment of the present disclosure. As shown in FIG. 7(a), a stent graft 710 can be provided within a wall of the thoracic aorta 720 (e.g., by using a wire and sheath). As shown in FIG. 7(b), a docking device 700 can be deployed (e.g., by using a wire and sheath, not shown) within a proximal end 710a of the stent graft 710, such that a bottom portion of the docking device 700 is flush or approximately flush with a bottom portion of the stent graft 710. Smaller caliber tubular structures 712 can be provided and a larger caliber tubular structure 714 can be provided within the docking device 700. As shown in FIG. 7(c), stent grafts 722, 724, 726 and 728 can be provided with one end in the tubular structures 712, and the other end in the walls of the visceral arteries 730, as described in FIGS. 5 and 6.

As shown in FIG. 7(d), a stent graft 740 is deployed and installed with a first end at or proximate to a distal end 710b of the stent graft 710, and a second end at the wall of the thoracic aorta 742. This can provide blood flow from the thoracic aorta 720 to the thoracic aorta 742, while preventing blood flow to the abdominal aortic aneurysm 750. Then, as shown in FIG. 7(e), a stent graft 752 can be installed such that a top portion expands into the larger caliber tubular structure 714 of the docking device 700. The stent graft 752 can have a first portion 754 that seals the wall of the iliac artery 758, and a second portion 756 that seals the wall of the iliac artery 762. In the embodiments described in FIGS. 7(a)-7(e), the docking device and stent grafts 710 and 740 can prevent blood flow to the abdominal aortic aneurysm 750, and the docking device 700 and stent grafts 722, 724, 726, 728 and 752 can prevent blood flow to the thoracoabdominal aortic aneurysm.

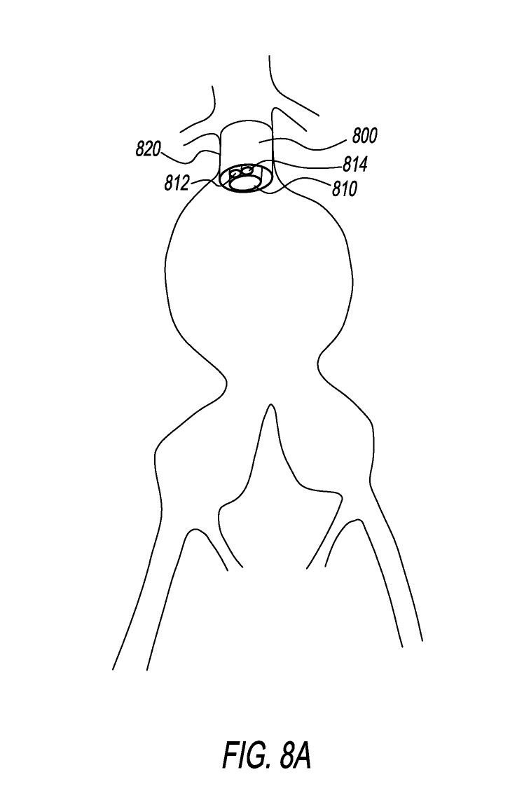

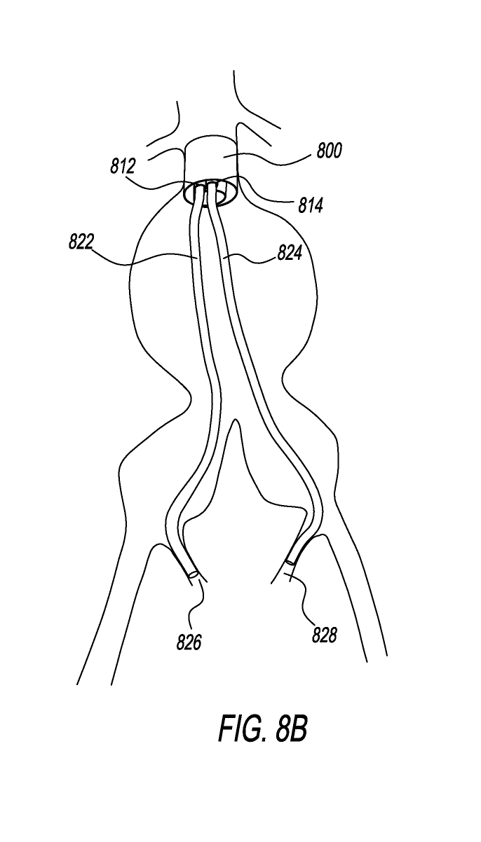

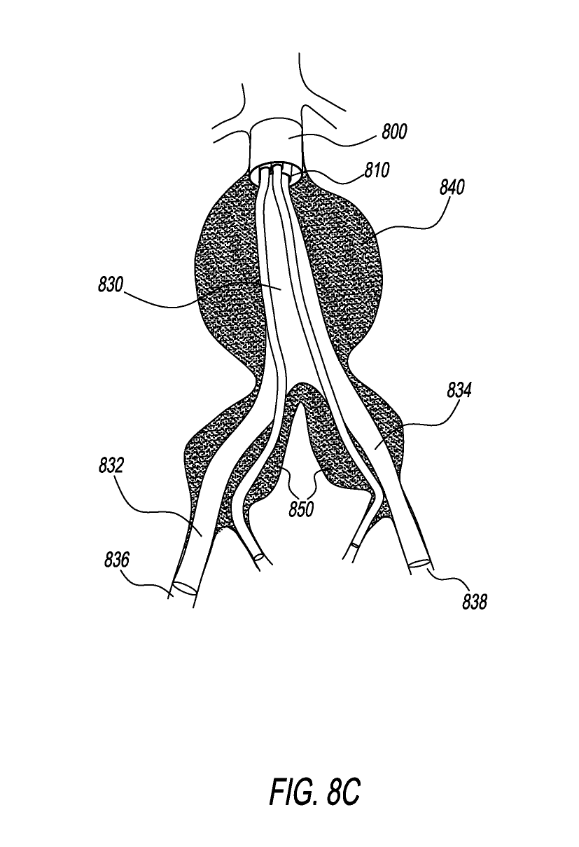

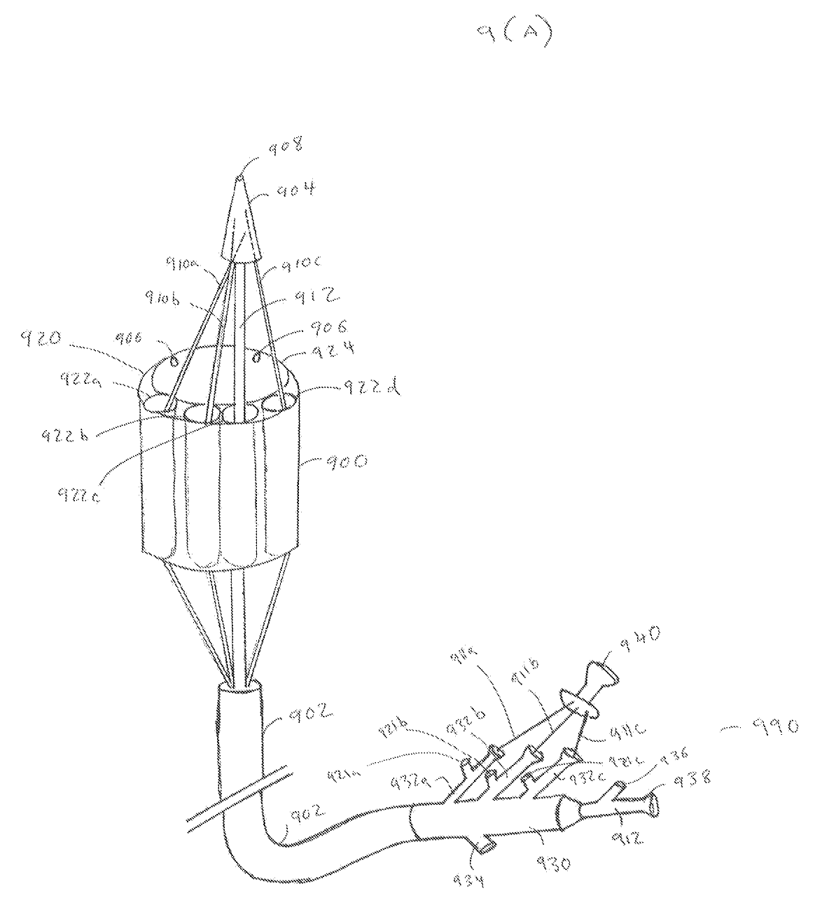

FIGS. 8(a)-8(c) illustrate a method of providing an endovascular dock to treat an abdominal aortic aneurysm and an iliac aortic aneurysm according to an exemplary embodiment of the present disclosure. Initially, as shown in FIG. 8(a), a docking device 800 is deployed and installed within an abdominal aortic wall 820, also known as the aortic neck above the aneurysm. The docking device 800 can have two smaller caliber tubular structures 812 and 814, and a larger caliber tubular structure 810. Then, as shown in FIG. 8(b), a stent graft 822 can be deployed and installed having a self-expanding top end within the smaller caliber tubular structure 812 (e.g., by using a wire and sheath), and a self-expanding bottom end to seal the wall of the internal iliac artery 826. A stent graft 824 can be deployed and installed having a self-expanding top end within the smaller caliber tubular structure 814 (e.g., by using a wire and sheath), and a self-expanding bottom end to seal the wall of the internal iliac artery 828.

Then, as shown in FIG. 8(c), a stent graft 830 can be deployed and installed having a self-expanding top end within the larger caliber tubular structure 810 (e.g., by using a wire and sheath). The stent graft 830 can have a first portion 832 and a second portion 834. The first portion 832 can have a self-expanding bottom end to seal the wall of the iliac artery 836, and the second portion 834 can have a self-expanding bottom end to seal the wall of the iliac artery 838. The docking device 800 and the stent grafts 822, 824 and 830 can prevent blood flow to the abdominal aortic aneurysm 840 and an iliac artery aneurysm 850, while allowing blood flow within the stent grafts and docking device.

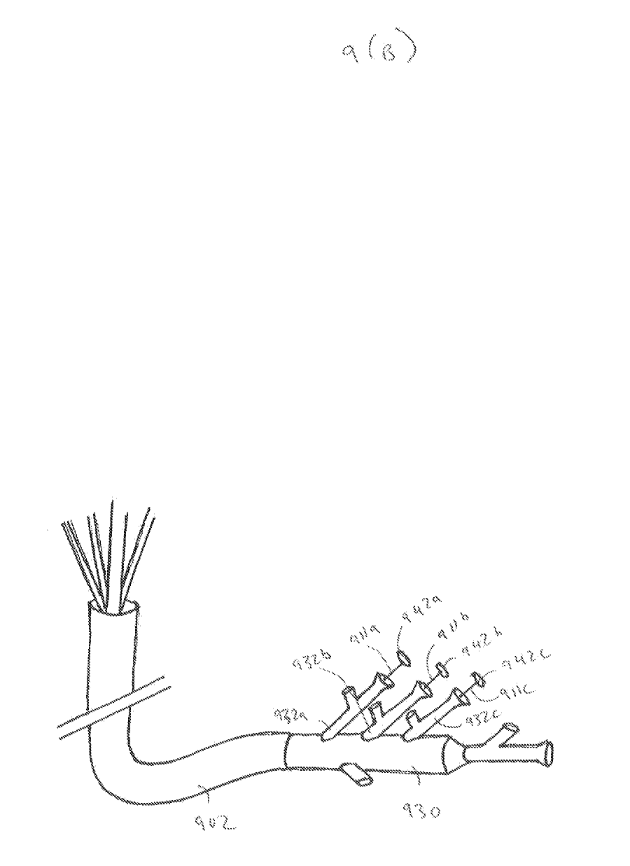

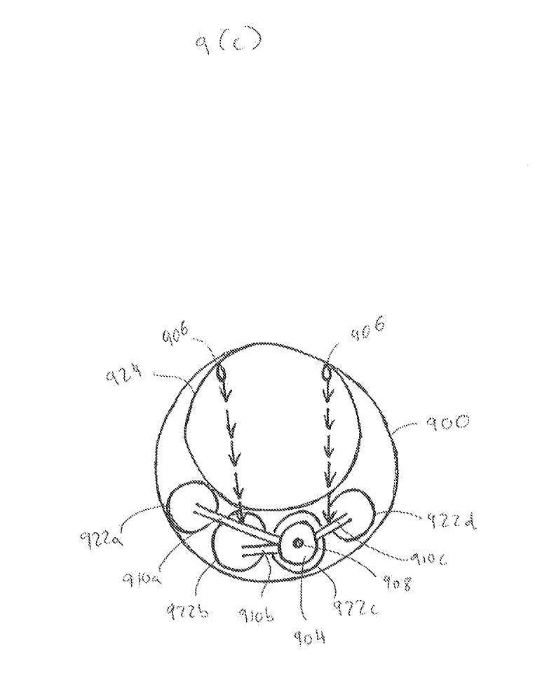

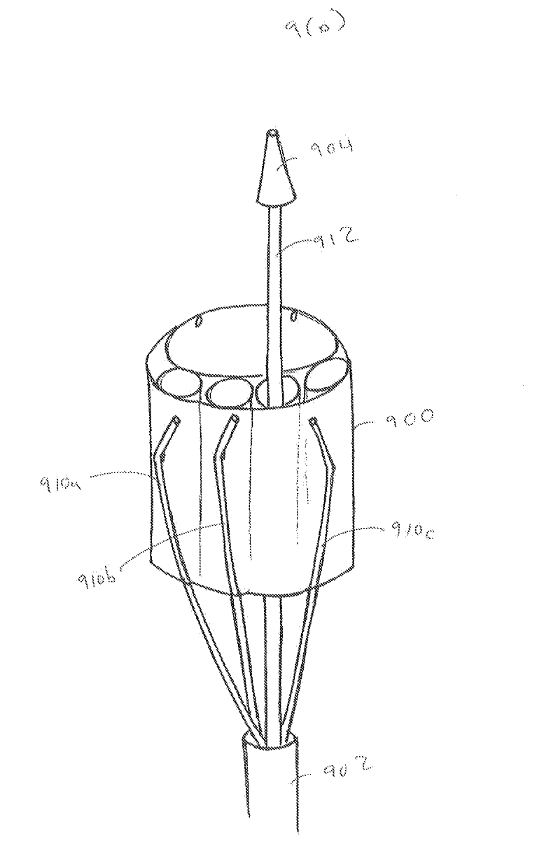

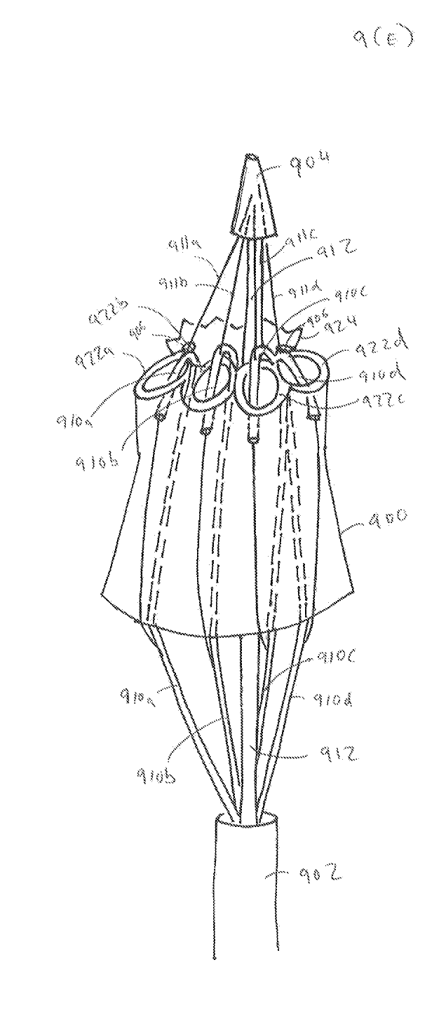

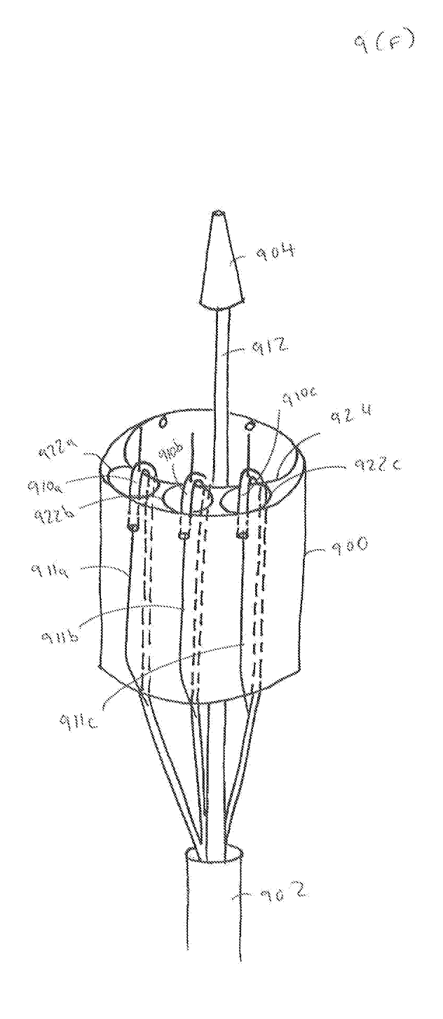

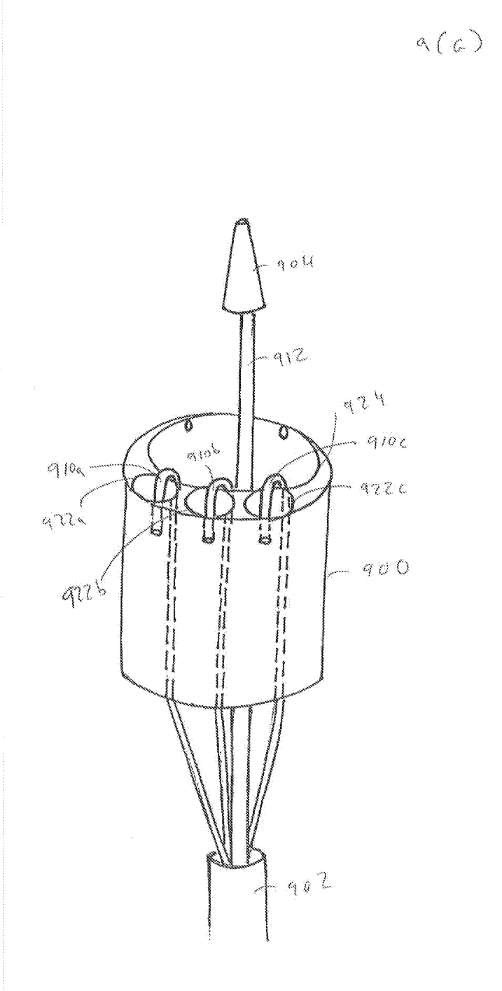

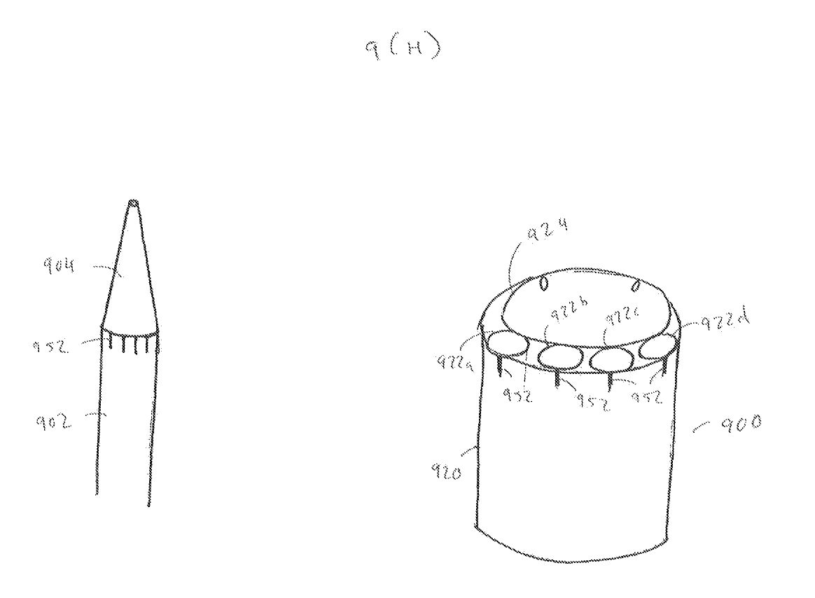

FIGS. 9(a)-9(d) illustrate a delivery system for delivering an endovascular dock according to an exemplary embodiment of the present disclosure. As shown in FIG. 9(a), an endovascular dock 900 can be provided within a blood vessel by using a delivery system, such as a sheath 902 and nose cone 904. This delivery system can provide for preloaded catheterization of the smaller tubular structures. The sheath 902 can be coated with a hydrophilic material. The sheath 902 can have a nose cone 904 at a top portion thereof. The sheath 902 can be preloaded with an endovascular dock 900 that is crimped and placed within the sheath 902. The nose cone 904 and sheath 902 can be mated with the endovascular dock 900 placed within the sheath 902. A guide wire (not shown) can be used (e.g., wire 402 in FIG. 4a) and then the nose cone 904 can be guided via the wire through port 938 and through the hole 908 in nose cone 904 for placement in the blood vessel. The sheath 902 and nose cone 904 can be guided via the wire. This particular delivery system can be used in, e.g., a descending thoracic aorta, as shown in FIGS. 5-8 above.

Once a location within the blood vessel is found, and the endovascular dock 900 is in place, the sheath 902 can be retracted at an operating end 990 so that the endovascular dock 900 is deployed within the blood vessel. The endovascular dock 900 can have an external tubular graft 920 having a larger tubular structure 924, and four smaller tubular structures 922a, 922b, 922c and 922d. The amount of tubular structures and sizes of tubular structures can vary, and in this particular embodiment one larger tubular structure and four smaller tubular structures are shown. This can vary depending on the need of the patient and location of the endovascular dock. Catheters 910a, 910b and 910c can be provided through three of the smaller tubular structures (here, 922a, 922b and 922d, respectively), extending from upper ports 932a, 932b and 932c, through the sheath 902 and into the nose cone 904 at an opposite end. Center shaft 912 can be provided through the sheath 902 and attached to the nose cone 904 at an opposite end. The center shaft 912 can be retractable within the inner sheath 930. Constraining guide wires 911a, 911b and 911c can be provided with one end attached to a mechanism, such as knob 940, through upper ports 932a, 932b and 932c, respectively, and through catheters 910a, 910b and 910c, respectively, into the nose cone 904.

An inner sheath 930 can be provided within the sheath 902 at an operating end 990. The knob 940 can allow for manipulation of the constraining guide wires 911a, 911b and 911c during delivery and placement of the endovascular dock 900. Flushing ports 921a, 921b and 921c can be provided in upper ports 932a, 932b and 932c, respectively. Flushing port 936 can be provided for side port 938, and flushing port 934 can be provided for inner sheath 930. The flushing ports can allow for flushing liquid (e.g., saline) or other solutions to lubricate the inside of the respective ports. The end port 938 can be provided to manipulate center shaft 912.

A valve mechanism can be provided within the sheath 902 to prevent bleeding. When the sheath 902 and nose cone 904 are in place, the inner sheath 930 can be held while sheath 902 is retracted so that the sheath 902 disengages from the nose cone 904, and the endovascular dock 900 is placed within the desired blood vessel. In some embodiments, as shown in FIG. 9(b), multiple knobs 942a, 942b and 942c can be provided for the constraining guide wires 911a, 911b and 911c, respectively, for manipulation of each guide wire separately.

As shown in FIG. 9(c), which illustrates a top view of the endovascular dock 900 in FIG. 9a, loops 906 can be provided on a top end inner lumen of the larger tubular structure 904. During packaging, the catheters 910a, 910c and constraining guide wires 911a, 911c, can be provided through these loops 906 so that the endovascular dock 900 is in a crimped position, enabling it to be packaged within the sheath 902. Once the constraining guide wires 911a and 911c are retracted, as will be described below, the guide wires 911a, 911c, as well as catheters 910a, 910c are retracted through the loop, allowing the endovascular dock 900 to expand (through stents provided on the tubular graft 920 or other mechanism).

As shown in FIG. 9(d), once the endovascular dock 900 is in an appropriate orientation, constraining guide wires 911a, 911b and 911c can be pulled back from the nose cone 904 an appropriate distance, allowing the catheters 910a, 910b and 910c, and constraining guide wires 911a, 911b and 911c to stay housed within the upper area of the smaller tubular structures 922a, 922b and 922d. This would release the endovascular dock 900 fully, and maintain catheter and constraining guide wire orientation within the smaller tubular structures 922a, 922b and 922d. The constraining guide wires 911a, 911b and 911c can then be fully retracted. The nose cone 904 and center shaft 910 can also then be fully retracted, and a guide wire can be used to place a catheter in smaller tubular structure 922c. These catheters in the smaller tubular structures can now be used to provide wire access through the smaller tubular structures for delivery of appropriately sized stent grafts for the blood vessels as needed, such as for the visceral arteries or the internal iliac arteries, as needed. The distal end (the end in the nose cone 904) of the catheters can be tapered, angled, straight or any other desired shape. The constraining guide wires can be any size, and can range from 0.014 to 0.038 inches, as needed, and can vary in size.

FIGS. 9(e)-9(g) illustrate another embodiment of a delivery system for delivering an endovascular dock according to an exemplary embodiment of the present disclosure. This embodiment could be used, e.g., for delivery of an endovascular dock in the ascending thoracic aorta (e.g., as shown in FIG. 4). Similar to FIGS. 9(a)-9(d), the nose cone 904 can be capped to the sheath 902 and provided via a guide wire to the desired location of the blood vessel, and then the sheath 902 can be withdrawn, and the preloaded endovascular dock 900 can be deployed. This provides a constrained deployment that still allows for rotation as well as forward and backward movement of the endovascular dock 900.

As shown in FIG. 9(e), a center shaft 912 is provided from the sheath 902 through the larger tubular structure 924, and attached to a nose cone 904 at an opposite end. Catheters 910a, 910b, 910c and 910d are provided from the sheath 902 through the larger tubular structure 924, and a top distal end provided within the smaller tubular structures 922a, 922b, 922c and 922d, respectively. The amount of tubular structures and sizes of tubular structures can vary, and in this particular embodiment one larger tubular structure and four smaller tubular structures are shown. This can vary depending on the need of the patient and location of the endovascular dock. The constraining guide wires 911a, 911b, 911c and 911d come through the catheters 910a, 910b, 910c and 910d from the sheath 902, and can puncture through the catheters 910a, 910b, 910c and 910d at an area below the endovascular dock 900. The constraining guide wires 911a, 911b, 911c and 911d run up the smaller tubular structures 922a, 922b, 922c and 922d, respectively, and into the top distal end of the catheters 910a, 910b, 910c and 910d, respectively. The constraining guide wires 911a, 911b, 911c and 911d can puncture a portion of the catheters 910a, 910b, 910c and 910d at an area above the endovascular dock 900 and are provided into the nose cone 904. Constraining guide wires 911a and 911d can be ensnared through the loops 906 before being provided into the nose cone.

As shown in FIG. 9(f), the constraining guide wires 911a, 911b and 911c (only three guide wires and catheters are shown for purposes of clarity) are pulled back from the nose cone 904 (e.g., at an operating end of the sheath 902, not shown), allowing the catheters 910a, 910b and 910c to stay housed within the upper portion of the smaller tubular structures 922a, 922b and 922c. The guide wires are removed from the loops 906, allowing the endovascular dock 900 to expand fully, and maintain catheter and constraining wire orientation within the smaller tubular structures. As shown in FIG. 9(g), the constraining guide wires 911a, 911b and 911c can then be withdrawn further from the distal end of the catheters 910a, 910b and 910c. These wires can subsequently be used for delivery of appropriately sized stent grafts for the blood vessels as needed (e.g., the visceral arteries or the internal iliac arteries).

Markers can be provided (e.g., radio opaque markings), on the catheters 910a, 910b, 910c and 910d within the smaller tubular structures 922a, 922b, 922c and 922d, respectively, and can be on the angled tips of the catheters, for ease of visualization as well as at a distal delivery sheath end to mark the amount of catheter with constraining wires that can be retracted back to fully release the endovascular dock 900 and maintain cannulation of the smaller tubular structures. Markings can be provided on the endovascular dock, catheters, and other elements as required so that any parts of the system can be viewed as may be necessary. The distal end of the catheters can be tapered, angled, straight or any other desired shape. The constraining guide wires can be any size, and can range from 0.014 to 0.038 inches, as needed, and can vary in size.

FIG. 9(h) illustrates an endovascular dock having radio opaque markings according to an exemplary embodiment of the present disclosure. The endovascular dock 900 can have radio opaque markings 952 along the external tubular graft 920, corresponding to the locations of the smaller tubular structures 922a, 922b, 922c and 922d. Accordingly, when the endovascular dock 900 is preloaded and placed within the sheath 902 (and capped by nose cone 904), it can be seen where the smaller tubular structures 922a, 922b, 922c and 922d are located and how many smaller tubular structures are contained within the endovascular dock 900 by using an x-ray.

Various methods and docking devices for treating aneurysms are contemplated by the present disclosure and are not limited to the embodiments described with reference to the figures. For example, the methods and systems of the present disclosure can be used to treat a wide variety of aneurysms, such as but not limited to visceral artery aneurysms, iliac artery aneurysms, femoral artery aneurysms, popliteal artery aneurysms, innominate artery aneurysms, subclavian artery aneurysms and/or carotid artery aneurysms, by, e.g., maintaining blood flow to certain parts of the body, such as arteries, and restricting blood flow to the aneurysms. Further, access for providing the docking device, wires, sheaths and stent grafts as described in the exemplary embodiments of the present disclosure can be provided from various parts of the body and the present disclosure is not limited to any particular point of access. Different methods of deployment can be provided for the docking device and the stent grafts, such as within a sheath, or using other methods or systems known to deploy stent grafts and similar devices.

The docking device of the exemplary embodiments of the present disclosure can also have various configurations. For example, the docking device can have a polymer, a polymer fill, ePTFE, polyethylene terephthalate, or any other suitable material that can be used to seal the openings between the tubular structures within the docking device to completely seal the blood flow to within the tubular structures. In some embodiments, the tubular structures can be configured to have no space between adjacent tubular structures such that a polymer or other suitable material is not needed. In some embodiments, the tubular structures can have a circular configuration while in other embodiments, the tubular structures can have other shapes. The docking device can have different sizes for its length and its diameter, as would be required for the particular application. The number of tubular structures within the docking device and the sizes of the tubular structures can be modified for the particular application required. For example, a different number of smaller tubular structures can be used in combination with a different number of larger tubular structures, and the exemplary embodiments of the present disclosure are not limited to any particular number or size. The size of the tubular structures, such as their length and diameter, can vary according to the particular application. The stent grafts used to connect the tubular structures to the arteries can vary in their length and type of stent graft used, such as self-expanding or fixed size stent grafts. The stent grafts can be connected to the tubular structures in various methods using various techniques, and the exemplary embodiments of the present disclosure are not limited to any particular type or size of stent graft.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, manufacture and methods which, although not explicitly shown or described herein, embody the principles of the disclosure and are thus within the spirit and scope of the disclosure. The disclosures of all documents and publications cited herein are hereby incorporated herein by reference in their entireties.

What is claimed is:

1. An apparatus for vascular surgery, comprising:
an external tubular graft capable of expansion and configured to be placed within a sheath in an unexpanded state;
a first tubular structure provided internally within the external tubular graft and configured for placement of a graft therein; and
a second tubular structure provided internally within the external tubular graft and configured for placement of a graft therein.

2. The apparatus of claim 1, wherein the external tubular graft comprises a fabric made of polytetrafluoroethylene or polyethylene terephthalate.

3. The apparatus of claim 1, further comprising:
one or more stents provide along a tubular wall of the external tubular graft.

4. The apparatus of claim 3, wherein the one or more stents are comprised of steel, nickel, titanium or nitinol.

5. The apparatus of claim 3, wherein the one or more stents are provided in one of a spiral, straight, circular or zigzag configuration.

6. The apparatus of claim 1, wherein the first tubular structure has a larger diameter than the second tubular structure.

7. The apparatus of claim 1, further comprising:
one or more stents provided along a tubular wall of the first tubular structure; and
one or more stents provided along a tubular wall of the second tubular structure.

8. The apparatus of claim 7, wherein the tubular wall of the first tubular structure and the tubular wall of the second tubular structure is attached to an inner portion of the tubular wall of the external tubular graft.

9. The apparatus of claim 1, wherein the first and second tubular structures have approximately a same height as the external tubular graft.

10. The apparatus of claim 1, further comprising:
a third tubular structure provided internally within the external tubular graft and configured for placement of a graft therein; and
a fourth tubular structure provided internally within the external tubular graft and configured for placement of a graft therein;
wherein the first tubular structure has a larger diameter than the second, third and fourth tubular structures, and the second, third and fourth tubular structures have approximately a same diameter.

11. A method of providing an apparatus for vascular surgery, comprising:
providing an external tubular stent graft having a tubular wall and configured to be placed within a sheath in an unexpanded state;
providing a first tubular structure within the external tubular stent graft and having a tubular wall attached to the tubular wall of the external tubular stent graft, and configured for placement of a graft therein; and
providing a second tubular structure within the external tubular stent graft and having a tubular wall attached to the tubular wall of the external tubular stent graft, and configured for placement of a graft therein.

12. The method of claim 11, further comprising:
providing stents on the tubular walls of the first and second tubular structures.

13. The method of claim 11, further comprising:
providing a third tubular structure within the external tubular stent graft and having a tubular wall attached to the tubular wall of the external tubular stent graft, and configured for placement of a graft therein; and
providing a fourth tubular structure within the external tubular stent graft and having a tubular wall attached to the tubular wall of the external tubular stent graft, and configured for placement of a graft therein.

14. The method of claim 13, wherein the first tubular structure has a larger diameter than the second, third and fourth tubular structures, and the second, third and fourth tubular structures have approximately a same diameter.

15. A method of performing vascular surgery, comprising:
providing an endovascular dock within a sheath, the endovascular dock comprising:
an external tubular stent graft having a tubular wall;
a first tubular structure provided within the tubular wall of the external tubular stent graft; and
a second tubular structure provided within the tubular wall of the external tubular stent graft;
retracting the sheath to dock the endovascular dock within a wall of a first blood vessel;
providing a first stent graft having a first end within the first tubular structure and a second end within a wall of a second blood vessel such that blood flow is substantially restricted to within the first stent graft between the first stent graft and the second blood vessel; and
providing a second stent graft having a first end within the second tubular structure and a second end within a wall of a third blood vessel to provide blood flow between the second stent graft and the third blood vessel such that blood flow is substantially restricted to within the second stent graft between the second stent graft and the third blood vessel.

16. The method of claim 15, further comprising:
providing a polymer to fill a void between the external walls of the first and second tubular structures and the internal wall of the external tubular stent graft of the endovascular dock.

17. The method of claim 15, wherein the first end of the first stent graft expands to conform to the shape of the first tubular structure and the second end of the first stent graft expands to conform to the shape of the wall of the second blood vessel, and the first end of the second stent graft expands to conform to the shape of the second tubular structure and the second end of the second stent graft expands to conform to the shape of the wall of the third blood vessel.

18. The method of claim 15, wherein the first stent graft is provided by obtaining access to the first tubular structure through the wall of the second blood vessel.

19. The method of claim 18, wherein the second stent graft is provided by obtaining access to the second tubular structure through the wall of the third blood vessel.

20. The method of claim 15, further comprising:
providing a third stent graft having a first end within the second end of the first stent graft, and a second end having a first and second tubular wall, a first tubular wall being provided within a wall of a fourth blood vessel such that blood flow is substantially restricted to between the first stent graft and the fourth blood vessel, and a second tubular wall being provided within a wall of a fifth blood vessel such that blood flow is substantially restricted to between the first stent graft and the fifth blood vessel.

21. A system for providing an endovascular dock within a blood vessel, comprising:
an endovascular dock having an external tubular stent graft;
a first tubular structure provided internally within the external tubular stent graft and configured for placement of a first graft therein;
a second tubular structure provided internally within the external tubular stent graft and configured for placement of a second graft therein;
a sheath for housing the endovascular dock within the sheath; and
a top portion connected to a distal end of the sheath;
wherein the endovascular dock is configured to be placed within a distal end of the sheath in a non-expanded state and is configured to expand when the sheath is retracted from the top portion.

22. The system of claim 21, wherein the top portion comprises a nose cone having a hole at a top portion for insertion of a wire.

23. The system of claim 21, further comprising:
a first catheter having one end within the sheath and extending through the first tubular structure into the top portion;
a second catheter having one end within the sheath and extending through the second tubular structure into the top portion;
a first guide wire provided within the first catheter;
a second guide wire provided within the second catheter; and
a center shaft provided having one end within the sheath and extending within the external tubular stent graft and attached to the top portion.

24. The system of claim 23, further comprising:
a third tubular structure provided internally within the external tubular stent graft and configured for placement of a third graft therein;
wherein the center shaft is provided through the third tubular structure within the external tubular stent graft.

25. The system of claim 21, further comprising:
one or more radio opaque markings along the external tubular stent graft in a location corresponding to the first and second tubular structures.
